(19) United States
(12) Patent Application Publication
(10) Pub. No.: US 2024/0350420 A1
(43) Pub. Date: Oct. 24, 2024

(54) PHARMACEUTICAL COMPOSITIONS AND METHODS FOR TREATING HYPERHIDROSIS

(71) Applicants: Wayne Stephen ANDREWS, Raleigh, NC (US); TheraVida, Inc., Chapel Hill, NC (US)

(72) Inventors: Stephen Wayne Andrews, Raleigh, NC (US); Samuel Bruce Balik, Raleigh, NC (US); John Edward Jett, Pittsboro, NC (US); Robert Michael Leming, Morganton, NC (US)

(21) Appl. No.: 18/294,487

(22) PCT Filed: Aug. 9, 2022
(86) PCT No.: PCT/US2022/039822

§ 371 Date: Feb. 1, 2024

Related U.S. Application Data
(60) Provisional application No. 63/260,154, filed on Aug. 11, 2021.

(51) Int. Cl. A61K 9/50; A61K 31/216; A61K 31/4178

(52) U.S. Cl. CPC A61K 9/5078; A61K 9/5084; A61K 31/216; A61K 31/4178

(57) ABSTRACT

Aspects of the disclosure include fixed dose pharmaceutical compositions including a plurality of immediate release beads of oxybutynin, or pharmaceutically acceptable salt thereof, beads and a plurality of delayed-immediate release beads of pilocarpine, or pharmaceutically acceptable salt thereof and methods for treating hyperhidrosis in a subject. In some embodiments the plurality of beads are encapsulated in a size 3 capsule. In practicing methods according to certain embodiments, a variety of formulations are described to achieve a delayed-immediate release profile wherein the pilocarpine, or pharmaceutically acceptable salt thereof, is released not more than 30% in 10 minutes and not less than 85% at 45 minutes of administration of the pharmaceutical composition to the subject and oxybutynin is released soon after administration.

### CROSS-REFERENCE TO RELATED APPLICATIONS

Pursuant to 35 U.S.C. § 119 (e), this application claims priority to the filing date of U.S. Provisional Patent Application Ser. No. 63/260,154 filed on Aug. 11, 2021; the disclosure of which application is incorporated herein by reference in its entirety.

### SUMMARY OF THE INVENTION

Sweat is an essential physiological function to human survival and serves as a body's coolant, protecting it from overheating. Eccrine glands secrete an odorless, clear fluid that helps the body to control its temperature by promoting heat loss through evaporation. Apocrine glands produce a thicker fluid which is often found in the armpits and near the genitals. Both the eccrine and apocrine sweat glands are activated by nerves.

Hyperhidrosis is a disorder characterized by an abnormal amount of sweating in excess of that required for regulation of body temperature. Hyperhidrosis can be either generalized or localized to specific parts of the body, including the hands, feet, armpits and genital region. It is estimated that 2-3% of Americans suffer from excessive sweating of the underarms (axillary hyperhidrosis), of the palms (palmar hyperhidrosis) or the soles of the feet (plantar hyperhidrosis). Prolonged hyperhidrosis can result in cold and clammy hands, dehydration as well as skin infections. However, most commonly subjects suffering from hyperhidrosis experience a significant quality of life burden from a psychological, emotional and social perspective, often modifying their lifestyles to accommodate the condition, which can lead to a disabling professional, academic, and social life.

Aspects of the disclosure include various pharmaceutical compositions comprising immediate release formulations of a therapeutically effective amount of oxybutynin and delayed-immediate release formulations of a therapeutically effective amount of pilocarpine and methods for treating hyperhidrosis in a subject with said pharmaceutical compositions. In embodiments, such a pharmaceutical composition is administered to a subject and is sufficient to reduce hyperhidrosis in the subject and to reduce a dry mouth side effect of the oxybutynin.

Pilocarpine is known to cause an increase in sweating. See, e.g., Salagen® (pilocarpine HCl) product insert (© 2003 MGI Pharma, Inc.). Accordingly, it was unexpected that the administration of a pharmaceutical composition including both oxybutynin and pilocarpine, according to the subject methods, would effectively treat hyperhidrosis while reducing a dry mouth side effect. Surprisingly, it was found that the pilocarpine neither diminished the efficacy of the oxybutynin in treating hyperhidrosis nor caused an increase in sweating in the subjects as is generally expected with the administration of pilocarpine.

However, developing a single pharmaceutical composition containing both a therapeutically effective amount of oxybutynin and a therapeutically effective amount of pilocarpine was challenging. It was discovered that the pilocarpine needed to be released about 30 minutes after the release of the oxybutynin to effectively treat hyperhidrosis and prevent the negative side effects, i.e. dry mouth, of oxybutynin. Accordingly, the pilocarpine was formulated into a delayed-immediate release bead. To administer both a therapeutically effective amount of oxybutynin and a therapeutically effective amount of pilocarpine in a single capsule, the oxybutynin was combined with the delayed-immediate release pilocarpine bead as a granulation. However, the formulation of an immediate release oxybutynin granulation with a delayed-immediate release pilocarpine bead presented numerous problems: 1) use of granulated oxybutynin can absorb water, especially in a high humidity environment; 2) filling of capsules becomes more complex wherein there may be non-uniform distribution in the capsule when granulated oxybutynin and pilocarpine beads are combined and 3) the size of the finished product was too large. There are multiple possibilities known to one skilled in the art to solve this problem, such as creating a compression coated tablet for each API, produce a single bilayer tablet comprising the immediate release API as the outer layer and the delayed-immediate release API as the inner layer, develop a single trilayer tablet containing a buffer layer to prevent APIs from interacting, or a capsule comprising multi-particulates of each API, to name a few.

As described here, an attempt to solve the above-mentioned problems, the oxybutynin was formulated as a bead, however that also required additional testing to determine how to formulate an immediate release oxybutynin bead with the necessary dissolution and pharmacokinetic profiles, but was small enough to fit together with the formulated delayed-immediate release pilocarpine beads in a capsule that would be suitable to orally administer to a human subject. During the development of the immediate release oxybutynin beads, it was discovered that there was a negative interaction between the oxybutynin and the excipient, HPMC, wherein processability was negatively affected.

The following embodiments described herein solve these problems and comprise fixed dose pharmaceutical compositions comprising an oxybutynin component and a pilocarpine component, wherein the oxybutynin component is a plurality of immediate release beads, each immediate release bead comprises: an inert core of the immediate release bead having a diameter of about 250 μm to about 700 μm, and a drug layer of the immediate release bead comprising oxybutynin, or a pharmaceutically acceptable salt thereof, coated on the inert core of the immediate release bead, and wherein the pilocarpine component is a plurality of delayed-immediate release beads, each delayed-immediate release bead comprises: an inert core of the delayed-immediate release bead having a diameter of about 250 μm to about 700 μm, a drug layer of the delayed-immediate release bead comprising pilocarpine, or a pharmaceutically acceptable salt thereof, coated on the inert core of the delayed-immediate release bead, and a delayed release coating of the delayed-immediate release bead coated on the drug layer of the delayed-immediate release bead, wherein the oxybutynin component contains about 2 mg to about 10 mg of oxybutynin, or pharmaceutically acceptable salt thereof, and wherein the pilocarpine component contains about 2 mg to about 10 mg of pilocarpine, or pharmaceutically acceptable salt thereof.

### DESCRIPTION OF THE DRAWINGS

FIG. 1 graphically illustrates the release profile of pilocarpine vs. % weight gain of the polymer layer.

FIG. 2 graphically compares the release profile of pilocarpine in a range of % weight polymer layer.

FIG. 3 graphically compares the release profile of oxybutynin in a granulation vs a formulation of beads.

FIG. 4 graphically compares the release profile of oxybutynin where PVP is used in the formulation of beads.

FIG. 5 represents the XRPD analysis of the oxybutynin/HPMC solid dispersion layered on MCC cellets.

FIG. 6 represents the dissolution profiles of (a) Immediate-release beads (b) Delayed-release beads with different levels of functional coating.

FIG. 7 graphically depicts the empirical model correlating % weight gain of functional coating with observed lag time (min) during in-vitro tests (a) and the relationship between % (ethylcellulose) weight gain, d50 (mm), and lag time (minutes) for end-point determination (b).

FIG. 8 represents the Dissolution Results for the Oxybutynin Cl Drug Layered Beads.

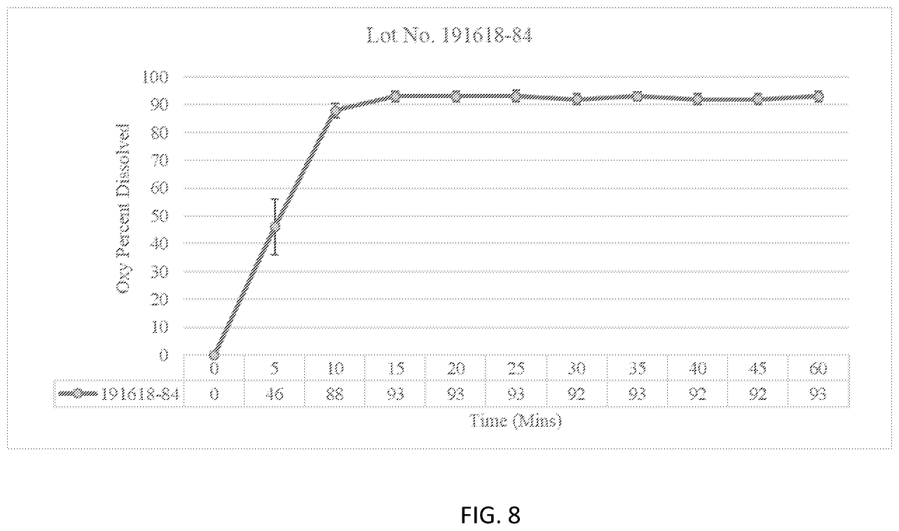

FIG. 9 represents the Dissolution Results for the Oxybutynin Cl Drug Layered Beads.

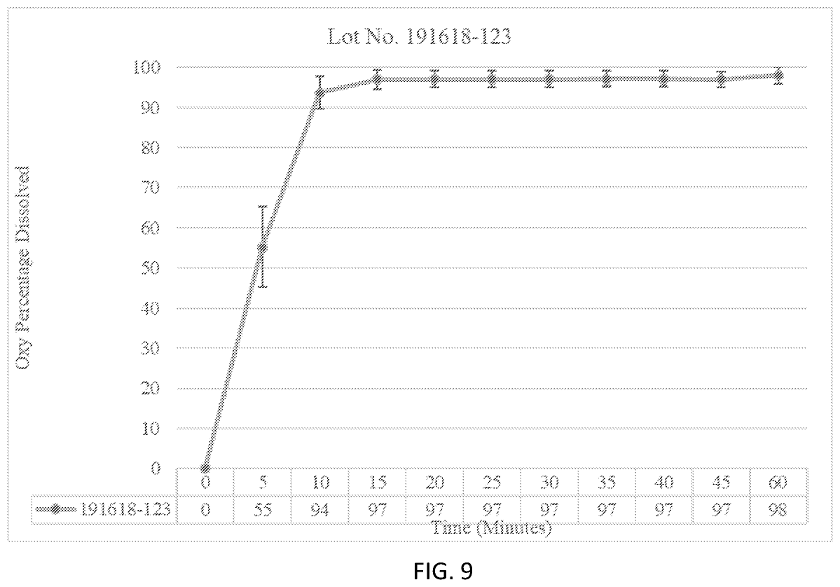

FIG. 10 represents the Dissolution Results for the Oxybutynin Cl Drug Layered Beads, Seal-Coated Clear.

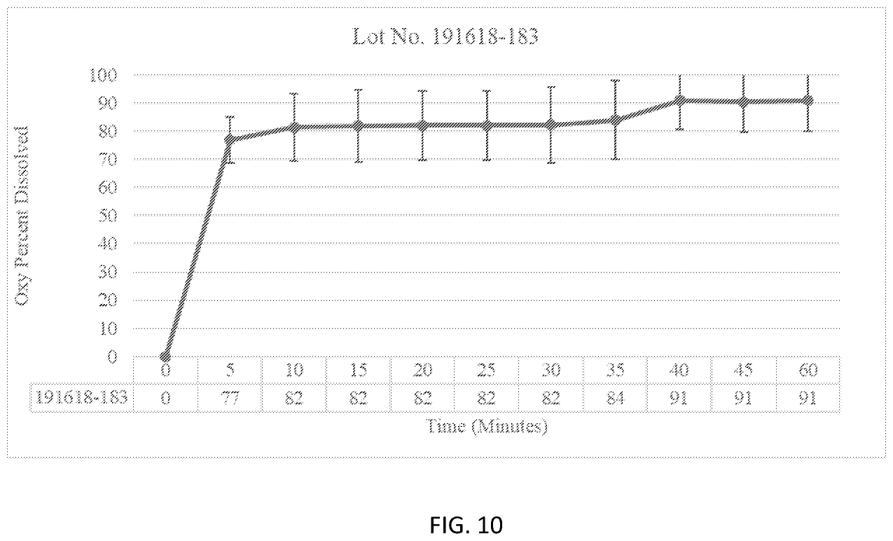

FIG. 11 represents the Dissolution Results for the Oxybutynin Cl Drug Layered Beads, Seal-Coated Clear.

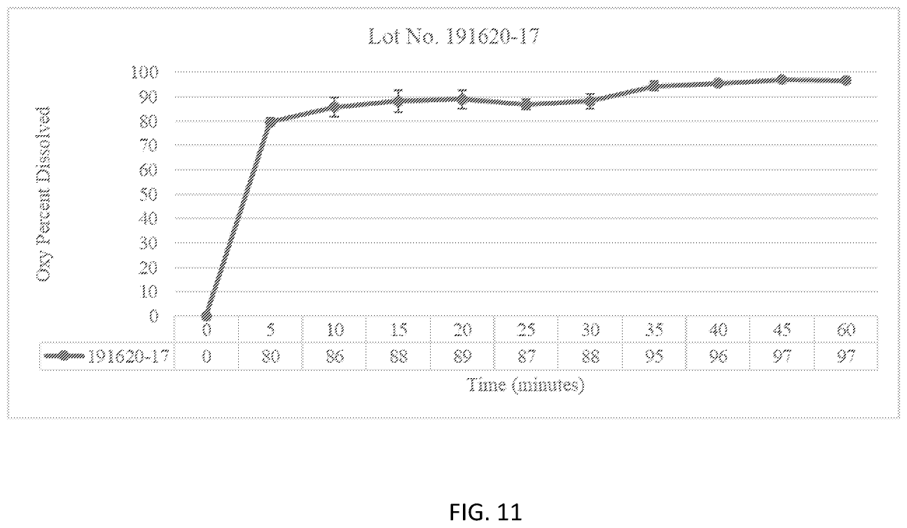

FIG. 12 represents the Dissolution Results for the Pilocarpine HCl DL Beads, Long Term Stability Batch No. 1.

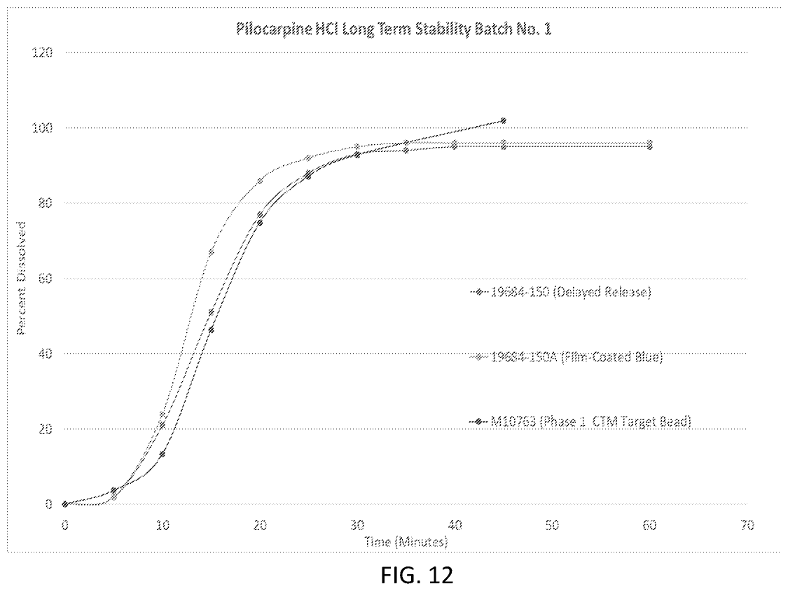

FIG. 13 represents the Dissolution Results for the Pilocarpine HCl DL Beads, Long Term Stability Batch No. 2.

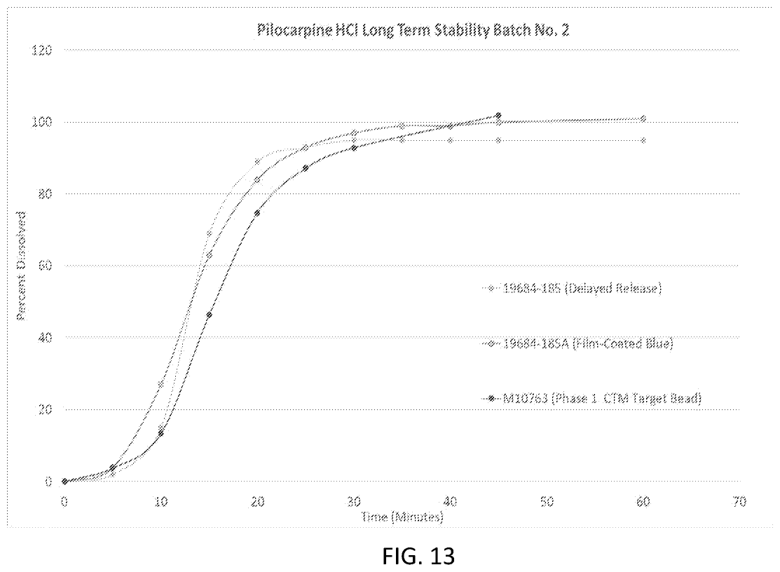

FIG. 14 is a comparison of Dissolution Profiles of Pilocarpine HCl Delayed-immediate Release Beads from Two API Vendors.

FIG. 15 is a comparison of Dissolution Results for the Pilocarpine HCl DL Beads Coated with a 3:1 EC/HPC at 20% Coating Weight Gain on 350 μm and 500 μm MCC Spheres.

FIG. 16 represents the effect of Coat Composition on Dissolution Rate.

FIG. 17 is the dissolution comparison of polymer layer at 3:1 ratio of EC:HPC coated at 12.5%, 15%, and 17.5% polymer layer coating levels.

FIG. 18 is the dissolution comparison of uncoated and seal coated Pilocarpine HCl delayed-immediate release beads.

FIG. 19 presents the bulk hold study dissolution profile for A) Oxybutynin Cl, 6 mg, and Pilocarpine HCl, 4 mg and B) Oxybutynin Cl, 6 mg, and Pilocarpine HCl, 8 mg.

FIG. 20 presents the initial dissolution profile for A) Pilocarpine HCl Capsules, 4 mg and B) Oxybutynin Cl Capsules, 6 mg.

FIG. 21 presents the dissolution profile for Capsules, Oxybutynin Cl, 6 mg and Pilocarpine HCl (C2), 4 mg at storage condition A) 25° C./60% RH, B) 30° C./75% RH, and C) 40° C./75% RH.

FIG. 22 presents the dissolution profile for Capsules, Oxybutynin Cl, 6 mg and Pilocarpine HCl (Iwaki), 8 mg at storage condition A) 25° C./60% RH, B) 30° C./75% RH, and C) 40° C./75% RH.

FIG. 23 presents the target dissolution profile for oxybutynin, or pharmaceutically acceptable salt thereof.

FIG. 24 presents the target dissolution profile for pilocarpine, or pharmaceutically acceptable salt thereof.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

It is noted that, as used herein and in the appended claims, the singular forms “a”, “an”, and “the” include plural referents unless the context clearly dictates otherwise. It is further noted that the claims may be drafted to exclude any recited element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating non-recited number may be a number which, in the context in which it is presented, provides the substantial equivalent of the specifically recited number. In some embodiments, the term “about”, when used to modify a value, encompasses a value that is within 10% of the value modified by the term “about”. The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. Furthermore, the phrases “less than about” a value or “greater than about” a value should be understood in view of the definition of the term “about” provided herein.

The terms “administer,” “administering” and “administration” as used herein refer to either directly administering a compound (also referred to as an agent of interest) or pharmaceutically acceptable salt of the compound (agent of interest) or a composition to a subject.

The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Therapeutically effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing or reducing the pathology and/or symptomatology).

The phrase “pharmaceutically acceptable” is employed herein to refer to those agents of interest/compounds, salts, compositions, dosage forms, etc., which are within the scope of sound medical judgment suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

As used herein, the term “pharmaceutically acceptable salt” is meant to indicate those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1-19, describes pharmaceutically acceptable salts in detail.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to slowing the progression of the disorder, disease or condition. In some embodiments, the term refers to alleviating the symptoms associated with the specific disorder, disease or condition. In some embodiments, the term refers to restoring function which was impaired or lost due to a specific disorder, disease or condition.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, representative illustrative methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided may be different from the actual publication dates which may need to be independently confirmed.

Embodiments are directed to fixed dose pharmaceutical compositions comprising an oxybutynin component and a pilocarpine component, wherein the oxybutynin component is a plurality of immediate release beads, each immediate release bead comprises: an inert core of the immediate release bead having a diameter of about 450 μm to about 700 μm, a drug layer of the immediate release bead comprising oxybutynin, or a pharmaceutically acceptable salt thereof, coated on the inert core of the immediate release bead, and, optionally, a seal coat coated on the delayed release coating, and wherein the pilocarpine component is a plurality of delayed-immediate release beads, each delayed-immediate release bead comprises: an inert core of the delayed-immediate release bead having a diameter of about 250 μm to about 550 μm, a drug layer of the delayed-immediate release bead comprising pilocarpine, or a pharmaceutically acceptable salt thereof, coated on the inert core of the delayed-immediate release bead, a delayed release coating of the delayed-immediate release bead coated on the drug layer of the delayed-immediate release bead, and, optionally, a seal coat coated on the delayed release coating, wherein the oxybutynin component contains about 2 mg to about 10 mg of oxybutynin, or pharmaceutically acceptable salt thereof, and wherein the pilocarpine component contains about 2 mg to about 10 mg of pilocarpine, or pharmaceutically acceptable salt thereof.

### Immediate Release Beads of Oxybutynin

In embodiments, a pharmaceutical composition comprises a plurality of immediate release beads, each immediate release bead comprises: an inert core of the immediate release bead having a diameter of about 450 μm to about 700 μm, a drug layer of the immediate release bead comprising oxybutynin, or a pharmaceutically acceptable salt thereof, coated on the inert core of the immediate release bead, and, optionally, a seal coat coated on the delayed release coating, wherein the total amount of oxybutynin, or pharmaceutically acceptable salt thereof, is a therapeutically effective amount—about 2 mg to about 10 mg—of the plurality of immediate release beads of the pharmaceutical composition. In preferred embodiments, the therapeutically effective amount of oxybutynin, or pharmaceutically acceptable salt thereof, is about 6 mg. In preferred embodiments, the therapeutically effective amount of oxybutynin, or pharmaceutically acceptable salt thereof, is about 4 mg. In preferred embodiments, the therapeutically effective amount of oxybutynin, or pharmaceutically acceptable salt thereof, is about 2 mg.

In certain embodiments, the oxybutynin, or pharmaceutically acceptable salt thereof, is in crystalline or amorphous form.

In certain embodiments, the inert core of the immediate release bead of oxybutynin, or pharmaceutically acceptable salt thereof, comprises a microcrystalline cellulose sphere (may also be referred to as MCC or cellets herein). In other embodiments, the inert core of the immediate release bead of oxybutynin, or pharmaceutically acceptable salt thereof, is selected from SUGLETS®, sugar spheres, tartaric acid pellets, and TAP®. In certain embodiments, the inert core of the immediate release bead is a microcrystalline cellulose sphere at about 70% to about 95% weight/weight of the immediate release bead. In certain embodiments, the inert core of the immediate release bead is a microcrystalline cellulose sphere at about 91.75% weight/weight of the immediate release bead. In certain embodiments, the inert core of the immediate release bead is a microcrystalline cellulose sphere at about 85.5% weight/weight of the immediate release bead. In certain embodiments, the inert core of the immediate release bead is a microcrystalline cellulose sphere at about 79.25% weight/weight of the immediate release bead. In certain embodiments, the inert core of the immediate release bead is a microcrystalline cellulose sphere at about 75% weight/weight of the immediate release bead. In certain embodiments, the inert core of the immediate release bead is a microcrystalline cellulose sphere at about 73.5% weight/weight of the immediate release bead. In certain embodiments, the inert core of the immediate release bead is a microcrystalline cellulose sphere at about 72.1% weight/weight of the immediate release bead.

In certain embodiments, the inert core of the immediate release bead of oxybutynin, or pharmaceutically acceptable salt thereof, comprises a microcrystalline cellulose sphere having a diameter of about 450 μm to about 700 μm, about 500 μm to about 650 μm, or about 550 μm to about 600 μm. Preferably, the inert core of the immediate release bead of oxybutynin, or pharmaceutically acceptable salt thereof, comprises a microcrystalline cellulose sphere having a diameter of about 500 μm.

In certain embodiments, the drug layer of the immediate release bead of oxybutynin, or pharmaceutically acceptable salt thereof, further comprises a binder, and an anti-tacking agent.

In certain embodiments, the drug layer of the immediate release bead of oxybutynin, or pharmaceutically acceptable salt thereof, comprises oxybutynin, or pharmaceutically acceptable salt thereof, at about 2% to about 15% weight/weight of the immediate release bead, the binder at about 2% to about 15% weight/weight of the immediate release bead, and the anti-tacking agent at about 1% to about 10% weight/weight of the immediate release bead. In certain embodiments, the drug layer of the immediate release bead of oxybutynin, or pharmaceutically acceptable salt thereof, comprises oxybutynin, or pharmaceutically acceptable salt thereof, at about 10% weight/weight of the immediate release bead, the binder at about 10% weight/weight of the immediate release bead, and the anti-tacking agent at about 5% weight/weight of the immediate release bead. In certain embodiments, the drug layer of the immediate release oxybutynin, or pharmaceutically acceptable salt thereof, bead comprises oxybutynin, or pharmaceutically acceptable salt thereof, at about 9.8% weight/weight of the immediate release bead, the binder at about 9.8% weight/weight of the immediate release bead, and the anti-tacking agent at about 4.9% weight/weight of the immediate release bead. In certain embodiments, the drug layer of the immediate release oxybutynin, or pharmaceutically acceptable salt thereof, bead comprises oxybutynin, or pharmaceutically acceptable salt thereof, at about 2.5% weight/weight of the immediate release bead, the binder at about 2.5% weight/weight of the immediate release bead, and the anti-tacking agent at about 1.25% weight/weight of the immediate release bead. In certain embodiments, the drug layer of the immediate release oxybutynin, or pharmaceutically acceptable salt thereof, bead comprises oxybutynin, or pharmaceutically acceptable salt thereof, at about 5% weight/weight of the immediate release bead, the binder at about 5% weight/weight of the immediate release bead, and the anti-tacking agent at about 2.5% weight/weight of the immediate release bead. In certain embodiments, the drug layer of the immediate release oxybutynin, or pharmaceutically acceptable salt thereof, bead comprises oxybutynin, or pharmaceutically acceptable salt thereof, at about 7.5% weight/weight of the immediate release bead, the binder at about 7.5% weight/weight of the immediate release bead, and the anti-tacking agent at about 3.75% weight/weight of the immediate release bead. In certain embodiments, the drug layer of the immediate release oxybutynin, or pharmaceutically acceptable salt thereof, bead comprises oxybutynin, or pharmaceutically acceptable salt thereof, at about 9.6% weight/weight of the immediate release bead, the binder at about 9.6% weight/weight of the immediate release bead, and the anti-tacking agent at about 4.8% weight/weight of the immediate release bead.

In certain embodiments, the therapeutically effective amount of oxybutynin, or a pharmaceutically acceptable salt thereof, in the pharmaceutical composition of a plurality of immediate release beads is about 2 mg to about 10 mg, about 2 mg to about 4 mg, about 2 mg to about 6 mg, or about 4 mg to about 6 mg. In certain embodiments, the therapeutically effective amount of oxybutynin, or a pharmaceutically acceptable salt thereof, in the pharmaceutical composition of a plurality of immediate release beads is about 2 mg. In certain embodiments, the therapeutically effective amount of oxybutynin, or a pharmaceutically acceptable salt thereof, in the pharmaceutical composition of a plurality of immediate release beads is about 4 mg. In certain embodiments, the therapeutically effective amount of oxybutynin, or a pharmaceutically acceptable salt thereof, in the pharmaceutical composition of a plurality of immediate release beads is about 8 mg. Most preferably, the therapeutically effective amount of oxybutynin, or a pharmaceutically acceptable salt thereof, in the pharmaceutical composition of a plurality of immediate release beads is about 6 mg.

In certain embodiments, the oxybutynin, or pharmaceutically acceptable salt thereof, to binder ratio in the immediate release bead is from 1:1 to 1.5:1. In certain embodiments, the oxybutynin, or pharmaceutically acceptable salt thereof, to binder ratio in the immediate release bead is from 1:1 to 2:1. In certain embodiments, the oxybutynin, or pharmaceutically acceptable salt thereof, to binder ratio in the immediate release bead is 5:1. In certain embodiments, the oxybutynin, or pharmaceutically acceptable salt thereof, to binder ratio in the immediate release bead is from 1:1 to 1:1.5. In certain embodiments, the oxybutynin, or pharmaceutically acceptable salt thereof, to binder ratio in the immediate release bead is from 1:1 to 1:2. In certain embodiments, the oxybutynin, or pharmaceutically acceptable salt thereof, to binder ratio in the immediate release bead is 1:1.

In embodiments described herein, the binder of the drug layer of the immediate release bead of oxybutynin, or pharmaceutically acceptable salt thereof, is selected from the group consisting of hydroxypropyl methylcellulose (HPMC), polyvinylpyrrolidone (PVP), povidone K 29/32, lactose, mannitol, sucrose, liquid glucose, acacia, tragacanth, gelatin, starch paste, pregelantinized starch, alginic acid, cellulose, methyl cellulose, ethyl cellulose, hydroxyl propyl cellulose (IPC), sodium carboxy methyl cellulose, polyethylene glycol (PEG), polyvinyl alcohol, hydroxyethyl cellulose (HEC), polyacrylates, polyvinyl alcohol (PVA), polymethacrylates, EUDRAGIT®, methylcellulose, cellulose acetate butyrates, and combinations thereof.

In embodiments described herein, the anti-tacking agent of the drug layer of the immediate release bead of oxybutynin, or pharmaceutically acceptable salt thereof, is selected from the group consisting of talc, magnesium stearate and other sterates, medium chain triglycerides (MCTs), talc, glyceryl dibehenate, silicon dioxide, and combinations thereof.

In certain embodiments, the drug layer of the immediate release bead of oxybutynin, or pharmaceutically acceptable salt thereof, comprises oxybutynin chloride, hydroxypropyl methylcellulose (HPMC) as the binder, and talc as the anti-tacking agent. In certain embodiments, the drug layer of the immediate release bead of oxybutynin, or pharmaceutically acceptable salt thereof, comprises oxybutynin chloride at about 2% to about 15% weight/weight of the immediate release bead, hydroxypropyl methylcellulose (HPMC) at about 2% to about 15% weight/weight of the immediate release bead, and talc at about 1% to about 10% weight/weight of the immediate release bead. In certain embodiments, the drug layer of the immediate release bead of oxybutynin, or pharmaceutically acceptable salt thereof, comprises oxybutynin chloride at about 10% weight/weight of the immediate release bead, hydroxypropyl methylcellulose (IPMC) at about 10% weight/weight of the immediate release bead, and talc at about 5% weight/weight of the immediate release bead. In certain embodiments, the drug layer of the immediate release bead of oxybutynin, or pharmaceutically acceptable salt thereof, comprises oxybutynin chloride at about 9.8% weight/weight of the immediate release bead, hydroxypropyl methylcellulose (IPMC) at about 9.8% weight/weight of the immediate release bead, and talc at about 4.9% weight/weight of the immediate release bead. In certain embodiments, the drug layer of the immediate release bead of oxybutynin, or pharmaceutically acceptable salt thereof, comprises oxybutynin chloride at about 2.5% weight/weight of the immediate release bead, hydroxypropyl methylcellulose (IPMC) at about 2.5% weight/weight of the immediate release bead, and talc at about 1.25% weight/weight of the immediate release bead. In certain embodiments, the drug layer of the immediate release bead of oxybutynin, or pharmaceutically acceptable salt thereof, comprises oxybutynin chloride at about 5% weight/weight of the immediate release bead, hydroxypropyl methylcellulose (IPMC) at about 5% weight/weight of the immediate release bead, and talc at about 2.5% weight/weight of the immediate release bead. In certain embodiments, the drug layer of the immediate release bead of oxybutynin, or pharmaceutically acceptable salt thereof, comprises oxybutynin chloride at about 7.5% weight/weight of the immediate release bead, hydroxypropyl methylcellulose (IPMC) at about 7.5% weight/weight of the immediate release bead, and talc at about 3.75% weight/weight of the immediate release bead.

In certain embodiments, each immediate release bead of oxybutynin, or pharmaceutically acceptable salt thereof, further comprises a seal coating coated on the drug layer of the immediate release bead. The seal coat may provide the following advantageous properties to each bead: 1. Use of different color seal coats distinguishes the two different beads, 2. Enhanced stability by providing an additional layer of separation between the drug layer and the other beads (and other API) in the capsule, 3. Protects manufacturing operator by minimizing exposure to the APIs, and 4. Mitigates static electricity during the encapsulation process. In certain embodiments, the seal coating of the immediate release bead is at about 0.5% to about 5.0% weight/weight of the immediate release bead. In certain embodiments, the seal coating of the immediate release bead is at about 1% to about 4.5%, about 1.5% to about 4.0%, about 2% to about 3.5%, about 2.5% to about 3.0%, about 1.0% to about 3%, about 1.0% to about 2.5%, or about 1.2% to about 2.0% weight/weight of the immediate release bead. In certain embodiments, the seal coating of the immediate release bead comprises about 2.0% weight/weight of the immediate release bead. In certain embodiments, the seal coating of the immediate release bead comprises about 3.8% weight/weight of the immediate release bead. In certain embodiments, the seal coating is a clear coat, such as any non-functional, commercially available coating, for example the Opadry® product line or their generic equivalents or shellac.

In some embodiments, each immediate release bead of oxybutynin, or pharmaceutically acceptable salt thereof, comprises 72.1% w/w 700 μm inert core, coated with a drug layer comprising 9.6% w/w oxybutynin chloride, 9.6% w/w hydroxypropyl methylcellulose (HPMC), and 4.8% w/w talc, and the drug layer is coated with 3.8% w/w of a seal coat. In some embodiments, each immediate release bead of oxybutynin, or pharmaceutically acceptable salt thereof, comprises 28.8 mg/dose 700 μm inert core, coated with a drug layer comprising 3.8 mg/dose oxybutynin chloride, 3.8 mg/dose hydroxypropyl methylcellulose (HPMC), and 1.9 mg/dose talc, and the drug layer is optionally coated with 1.6 mg/dose of a seal coat. In some embodiments, each immediate release bead of oxybutynin, or pharmaceutically acceptable salt thereof, comprises 43.3 mg/dose 700 μm inert core, coated with a drug layer comprising 5.8 mg/dose oxybutynin chloride, 3.8 mg/dose hydroxypropyl methylcellulose (HPMC), and 2.9 mg/dose talc, and the drug layer is coated with 2.4 mg/dose of a seal coat. In some embodiments, each immediate release bead of oxybutynin, or pharmaceutically acceptable salt thereof, comprises 57.7 mg/dose 700 μm inert core, coated with a drug layer comprising 7.7 mg/dose oxybutynin chloride, 3.8 mg/dose hydroxypropyl methylcellulose (HPMC), and 3.8 mg/dose talc, and the drug layer is coated with 3.2 mg/dose of a seal coat.

In some embodiments, each immediate release bead of oxybutynin, or pharmaceutically acceptable salt thereof, comprises 75% w/w 700 μm inert core, coated with a drug layer comprising 10% w/w oxybutynin chloride, 10% w/w hydroxypropyl methylcellulose (HPMC), and 5% w/w talc. In some embodiments, each immediate release bead of oxybutynin, or pharmaceutically acceptable salt thereof, comprises 56.25 mg/dose 700 μm inert core, coated with a drug layer comprising 7.5 mg/dose oxybutynin chloride, 7.5 mg/dose hydroxypropyl methylcellulose (HPMC), and 3.75 mg/dose talc. In embodiments described herein, the total fill per dose of a plurality of immediate release beads of oxybutynin, or pharmaceutically acceptable salt thereof, is 75 mg.

In some embodiments, each immediate release bead of oxybutynin, or pharmaceutically acceptable salt thereof, comprises 91.75% w/w 500 μm inert core, coated with a drug layer comprising 2.5% w/w oxybutynin chloride, 2.5% w/w hydroxypropyl methylcellulose (HPMC), and 1.25% w/w talc, and the drug layer is coated with 2% w/w of a seal coat. In some embodiments, each immediate release bead of oxybutynin, or pharmaceutically acceptable salt thereof, comprises 73.4 mg/dose 500 μm inert core, coated with a drug layer comprising 2.0 mg/dose oxybutynin chloride, 2.0 mg/dose hydroxypropyl methylcellulose (HPMC), and 1.0 mg/dose talc, and the drug layer is coated with 1.6 mg/dose of a seal coat. In embodiments described herein, the total fill per dose of a plurality of immediate release beads of oxybutynin, or pharmaceutically acceptable salt thereof, is 80 mg.

In some embodiments, each immediate release bead of oxybutynin, or pharmaceutically acceptable salt thereof, comprises 85.5% w/w 500 μm inert core, coated with a drug layer comprising 5% w/w oxybutynin chloride, 5% w/w hydroxypropyl methylcellulose (HPMC), and 2.5% w/w talc, and the drug layer is coated with 2% w/w of a seal coat. In some embodiments, each immediate release bead of oxybutynin, or pharmaceutically acceptable salt thereof, comprises 68.4 mg/dose 500 μm inert core, coated with a drug layer comprising 4.0 mg/dose oxybutynin chloride, 4.0 mg/dose hydroxypropyl methylcellulose (HPMC), and 2.0 mg/dose talc, and the drug layer is coated with 1.6 mg/dose of a seal coat. In embodiments described herein, the total fill per dose of a plurality of immediate release beads of oxybutynin, or pharmaceutically acceptable salt thereof, is 80 mg.

In some embodiments, each immediate release bead of oxybutynin, or pharmaceutically acceptable salt thereof, comprises 79.25% w/w 500 μm inert core, coated with a drug layer comprising 7.5% w/w oxybutynin chloride, 7.5% w/w hydroxypropyl methylcellulose (HPMC), and 3.75% w/w talc, and the drug layer is coated with 2% w/w of a seal coat. In some embodiments, each immediate release bead of oxybutynin, or pharmaceutically acceptable salt thereof, comprises 63.4 mg/dose 500 μm inert core, coated with a drug layer comprising 6.0 mg/dose oxybutynin chloride, 6.0 mg/dose hydroxypropyl methylcellulose (HPMC), and 3.0 mg/dose talc, and the drug layer is coated with 1.6 mg/dose of a seal coat. In embodiments described herein, the total fill per dose of a plurality of immediate release beads of oxybutynin, or pharmaceutically acceptable salt thereof, is 80 mg.

In some embodiments, each immediate release bead of oxybutynin, or pharmaceutically acceptable salt thereof, comprises 73.5% w/w 500 μm inert core, coated with a drug layer comprising 9.8% w/w oxybutynin chloride, 9.8% w/w hydroxypropyl methylcellulose (HPMC), and 4.9% w/w talc, and the drug layer is coated with 2% w/w of a seal coat. In some embodiments, each immediate release bead of oxybutynin, or pharmaceutically acceptable salt thereof, comprises 60 mg/dose 500 μm inert core, coated with a drug layer comprising 8.0 mg/dose oxybutynin chloride, 8.0 mg/dose hydroxypropyl methylcellulose (HPMC), and 4.0 mg/dose talc, and the drug layer is coated with 1.6 mg/dose of a seal coat. In embodiments described herein, the total fill per 8 mg dose of a plurality of immediate release beads of oxybutynin, or pharmaceutically acceptable salt thereof, is 81.6 mg. In embodiments described herein, the total fill per 6 mg dose of a plurality of immediate release beads of oxybutynin, or pharmaceutically acceptable salt thereof, is 61.2 mg. In embodiments described herein, the total fill per 4 mg dose of a plurality of immediate release beads of oxybutynin, or pharmaceutically acceptable salt thereof, is 40.8 mg.

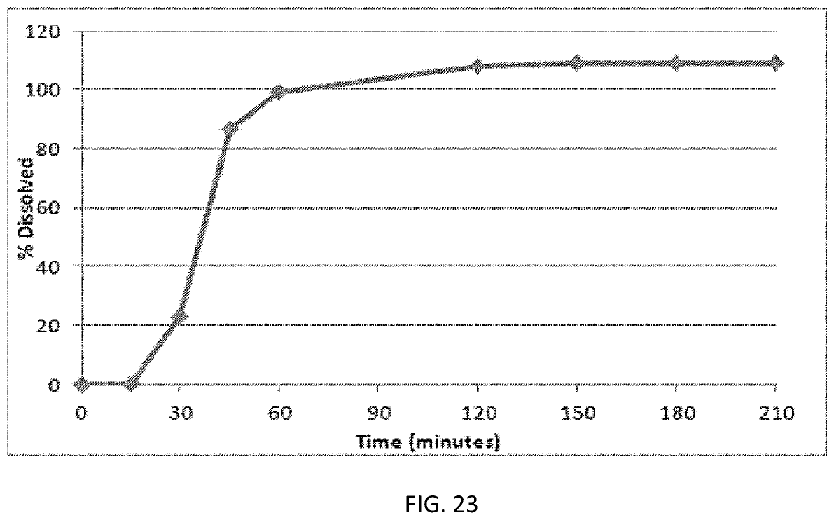

Embodiments described herein are directed to immediate release beads of oxybutynin, or pharmaceutically acceptable salt thereof, having a target dissolution profile which is equivalent to the Cₘₐₓ, Tₘₐₓ and AUC. The target dissolution profile is measured in vitro in 0.1N HCl as depicted in FIG. 23. In some embodiments, the AUC of the immediate release beads of oxybutynin, or pharmaceutically acceptable salt thereof, is about 8 ng*hr/ml to about 12 ng*hr/ml. In some embodiments, the Cₘₐₓ of the immediate release beads of oxybutynin, or pharmaceutically acceptable salt thereof, is about 3 ng/ml to about 6 ng/ml. In some embodiments, the Tₘₐₓ of the immediate release beads of oxybutynin, or pharmaceutically acceptable salt thereof, is about 0.75 hour to about 1 hour.

In some embodiments, the plurality of immediate release beads of oxybutynin, or a pharmaceutically acceptable salt thereof, release about 50% or more, about 60% or more, about 75% or more, about 90% or more, about 95% or more, about 99% or more, or 100% of oxybutynin, or a pharmaceutically acceptable salt thereof, within about 10 minutes or less of administration of the pharmaceutical composition to a subject. In some embodiments, the plurality of immediate release beads of oxybutynin, or a pharmaceutically acceptable salt thereof, release about 50% or more, about 60% or more, about 75% or more, about 90% or more, about 95% or more, about 99% or more, or 100% of oxybutynin, or a pharmaceutically acceptable salt thereof, within about 5 minutes or less of administration of the pharmaceutical composition to a subject. In some embodiments, the plurality of immediate release beads of oxybutynin, or a pharmaceutically acceptable salt thereof, release about 50% or more, about 60% or more, about 75% or more, about 90% or more, about 95% or more, about 99% or more, or 100% of oxybutynin, or a pharmaceutically acceptable salt thereof, immediately after administration of the pharmaceutical composition to a subject.

In some embodiments, the plurality of immediate release beads of oxybutynin, or a pharmaceutically acceptable salt thereof, is stable at 25° C./60% relative humidity (RH) for at least 4 weeks, at least 8 weeks, at least 12 weeks, at least 16 weeks, at least 20 weeks, or at least 24 weeks. In some embodiments, the plurality of immediate release beads of oxybutynin, or a pharmaceutically acceptable salt thereof, is stable at 25° C./60% relative humidity (RH) for at least 1 month, at least 3 months, at least 6 months, at least 9 months, at least 12 months, or at least 18 months. In some embodiments, the plurality of immediate release beads of oxybutynin, or a pharmaceutically acceptable salt thereof, is stable at 30° C./75% RH for at least 4 weeks, at least 8 weeks, at least 12 weeks, at least 16 weeks, at least 20 weeks, or at least 24 weeks. In some embodiments, the plurality of immediate release beads of oxybutynin, or a pharmaceutically acceptable salt thereof, is stable at 40° C./75% RH for at least 4 weeks, at least 8 weeks, at least 12 weeks, at least 16 weeks, at least 20 weeks, or at least 24 weeks. In some embodiments, the plurality of immediate release beads of oxybutynin, or a pharmaceutically acceptable salt thereof, is stable at 40° C./75% RH for at least 1 month, at least 3 months, or at least 6 months. In some embodiments, the plurality of immediate release beads of oxybutynin, or a pharmaceutically acceptable salt thereof, is stable at 50° C./ambient conditions (AMB) for at least 4 weeks, at least 8 weeks, at least 12 weeks, at least 16 weeks, at least 20 weeks, or at least 24 weeks.

### Delayed-Immediate Release Beads of Pilocarpine

In embodiments described herein, a pharmaceutical composition comprises a plurality of delayed-immediate release beads, each delayed-immediate release bead comprises: an inert core of the delayed-immediate release bead having a diameter of about 250 μm to about 700 μm, a drug layer of the delayed-immediate release bead comprising pilocarpine, or a pharmaceutically acceptable salt thereof, coated on the inert core of the delayed-immediate release bead, a delayed release coating of the delayed-immediate release bead coated on the drug layer of the delayed-immediate release bead, and, optionally, a seal coat coated on the delayed release coating, wherein the total amount of pilocarpine, or pharmaceutically acceptable salt thereof, is a therapeutically effective amount—about 2 mg to about 10 mg—of the plurality of delayed-immediate release beads of the pharmaceutical composition. In preferred embodiments, the therapeutically effective amount of pilocarpine, or pharmaceutically acceptable salt thereof, is about 4 mg. In other preferred embodiments, the therapeutically effective amount of pilocarpine, or pharmaceutically acceptable salt thereof, is about 8 mg.

In embodiments, the pilocarpine, or pharmaceutically acceptable salt thereof, is in crystalline or amorphous form.

In certain embodiments, the inert core of the delayed-immediate release bead of pilocarpine, or pharmaceutically acceptable salt thereof, comprises a microcrystalline cellulose sphere (may also be referred to as MCC or cellets herein). In other embodiments, the inert core of the delayed-immediate release bead of pilocarpine, or pharmaceutically acceptable salt thereof, is selected from SUGLETS®, sugar spheres, tartaric acid pellets, and TAP®. In certain embodiments, the inert core of the delayed-immediate release bead is a microcrystalline cellulose sphere at about 10% to about 75% weight/weight of the delayed-immediate release bead. In certain embodiments, the inert core of the delayed-immediate release bead is a microcrystalline cellulose sphere at about 71.69% weight/weight of the delayed-immediate release bead. In certain embodiments, the inert core of the delayed-immediate release bead is a microcrystalline cellulose sphere at about 31.25% weight/weight of the delayed-immediate release bead. In certain embodiments, the inert core of the delayed-immediate release bead is a microcrystalline cellulose sphere at about 26.32% weight/weight of the delayed-immediate release bead. In certain embodiments, the inert core of the delayed-immediate release bead is a microcrystalline cellulose sphere at about 20.83% weight/weight of the delayed-immediate release bead. In certain embodiments, the inert core of the delayed-immediate release bead is a microcrystalline cellulose sphere at about 17.86% weight/weight of the delayed-immediate release bead. In certain embodiments, the inert core of the delayed-immediate release bead is a microcrystalline cellulose sphere at about 14.71% weight/weight of the delayed-immediate release bead.

In certain embodiments, the inert core of the delayed-immediate release bead of pilocarpine, or pharmaceutically acceptable salt thereof, comprises microcrystalline cellulose sphere having a diameter of about 250 μm to about 700 μm, about 300 μm to about 650 μm, about 350 μm to about 600 μm, or about 400 μm to about 500 μm. In certain embodiments, the inert core of the delayed-immediate release bead of pilocarpine, or pharmaceutically acceptable salt thereof, comprises a microcrystalline cellulose sphere having a diameter of about 500 μm.

In certain embodiments, the drug layer of the delayed-immediate release bead of pilocarpine, or pharmaceutically acceptable salt thereof, further comprises a binder and an anti-tacking agent.

In certain embodiments, the drug layer of the delayed-immediate release bead of pilocarpine, or pharmaceutically acceptable salt thereof, comprises pilocarpine, or pharmaceutically acceptable salt thereof, at about 5% to about 15% weight/weight of the delayed-immediate release bead, the binder at about 1% to about 15% weight/weight of the delayed-immediate release bead, and the anti-tacking agent at about 1% to about 10% weight/weight of the delayed-immediate release bead. In certain embodiments, the drug layer of the delayed-immediate release bead of pilocarpine, or pharmaceutically acceptable salt thereof, comprises pilocarpine, or pharmaceutically acceptable salt thereof, at about 8.43% weight/weight of the delayed-immediate release bead, the binder at about 1.69% weight/weight of the delayed-immediate release bead, and the anti-tacking agent at about 2.53% weight/weight of the delayed-immediate release bead. In certain embodiments, the drug layer of the delayed-immediate release bead of pilocarpine, or pharmaceutically acceptable salt thereof, comprises pilocarpine, or pharmaceutically acceptable salt thereof, at about 12.5% weight/weight of the delayed-immediate release bead, the binder at about 12.5% weight/weight of the delayed-immediate release bead, and the anti-tacking agent at about 6.25% weight/weight of the delayed-immediate release bead. In certain embodiments, the drug layer of the delayed-immediate release bead of pilocarpine, or pharmaceutically acceptable salt thereof, comprises pilocarpine, or pharmaceutically acceptable salt thereof, at about 10.53% weight/weight of the delayed-immediate release bead, the binder at about 10.53% weight/weight of the delayed-immediate release bead, and the anti-tacking agent at about 5.26% weight/weight of the delayed-immediate release bead. In certain embodiments, the drug layer of the delayed-immediate release bead of pilocarpine, or pharmaceutically acceptable salt thereof, comprises pilocarpine, or pharmaceutically acceptable salt thereof, at about 8.33% weight/weight of the delayed-immediate release bead, the binder at about 8.33% weight/weight of the delayed-immediate release bead, and the anti-tacking agent at about 4.17% weight/weight of the delayed-immediate release bead. In certain embodiments, the drug layer of the delayed-immediate release bead of pilocarpine, or pharmaceutically acceptable salt thereof, comprises pilocarpine, or pharmaceutically acceptable salt thereof, at about 7.14% weight/weight of the delayed-immediate release bead, the binder at about 7.14% weight/weight of the delayed-immediate release bead, and the anti-tacking agent at about 3.57% weight/weight of the delayed-immediate release bead. In certain embodiments, the drug layer of the delayed-immediate release bead of pilocarpine, or pharmaceutically acceptable salt thereof, comprises pilocarpine, or pharmaceutically acceptable salt thereof, at about 5.88% weight/weight of the delayed-immediate release bead, the binder at about 5.88% weight/weight of the delayed-immediate release bead, and the anti-tacking agent at about 2.94% weight/weight of the delayed-immediate release bead.

In certain embodiments, the therapeutically effective amount of pilocarpine, or a pharmaceutically acceptable salt thereof, in the pharmaceutical composition of a plurality of delayed-immediate release beads is about 2 mg to about 10 mg, about 2 mg to about 4 mg, about 2 mg to about 6 mg, or about 4 mg to about 6 mg. In certain embodiments, the therapeutically effective amount of pilocarpine, or a pharmaceutically acceptable salt thereof, in the pharmaceutical composition of a plurality of delayed-immediate release beads is about 4 mg. In certain embodiments, the therapeutically effective amount of pilocarpine, or a pharmaceutically acceptable salt thereof, in the pharmaceutical composition of a plurality of delayed-immediate release beads is about 6 mg. In certain embodiments, the therapeutically effective amount of pilocarpine, or a pharmaceutically acceptable salt thereof, is about 8 mg in the pharmaceutical composition of a plurality of delayed-immediate release beads.

In certain embodiments, the pilocarpine, or pharmaceutically acceptable salt thereof, to binder ratio in the delayed-immediate release bead is from 1:1 to 5:1. In certain embodiments, the pilocarpine, or pharmaceutically acceptable salt thereof, to binder ratio in the delayed-immediate release bead is from 1:1 to 2.5:1. In certain embodiments, the pilocarpine, or pharmaceutically acceptable salt thereof, to binder ratio the delayed-immediate release bead is 5:1. In certain embodiments, the pilocarpine, or pharmaceutically acceptable salt thereof, to binder ratio in the delayed-immediate release bead is from 1:1 to 1:5. In certain embodiments, the pilocarpine, or pharmaceutically acceptable salt thereof, to binder ratio in the delayed-immediate release bead is from 1:1 to 1:2.5. In certain embodiments, the pilocarpine, or pharmaceutically acceptable salt thereof, to binder ratio the delayed-immediate release bead is 1:1. Preferably, the drug substance to binder ratio in the delayed-immediate release pilocarpine bead is 5:1.

In embodiments described herein, the binder of the drug layer of the delayed-immediate release bead of pilocarpine, or pharmaceutically acceptable salt thereof, is selected from the group consisting of hydroxypropyl methylcellulose (HPMC), polyvinylpyrrolidone (PVP), povidone K 29/32, lactose, mannitol, sucrose, liquid glucose, acacia, tragacanth, gelatin, starch paste, pregelantinized starch, alginic acid, cellulose, methyl cellulose, ethyl cellulose, hydroxyl propyl methyl cellulose (HPMC), hydroxyl propyl cellulose (HPC), sodium carboxy methyl cellulose, polyvinyl pyrrolidone (PVP), polyethylene glycol (PEG), polyvinyl alcohol, hydroxyethyl cellulose (HEC), polyacrylates, polyvinyl alcohol (PVA), polymethacrylates, methyl methacrylate (EUDRAGIT®), methylcellulose, cellulose acetate butyrates, and combinations thereof.

In embodiments described herein, the anti-tacking agent of the drug layer of the delayed-immediate release bead of pilocarpine, or pharmaceutically acceptable salt thereof, is selected from the group consisting of talc, magnesium stearate and other sterates, stearic acid, medium chain triglycerides (MCTs), talc, glyceryl dibehenate, silicon dioxide, and combinations thereof.

In certain embodiments, the drug layer of the delayed-immediate release bead of pilocarpine, or pharmaceutically acceptable salt thereof, comprises pilocarpine hydrochloride, hydroxypropyl methylcellulose (HPMC) as the binder, and talc as the anti-tacking agent. In certain embodiments, the drug layer of the delayed-immediate release bead of pilocarpine, or pharmaceutically acceptable salt thereof, comprises pilocarpine hydrochloride at about 5% to about 15% weight/weight of the delayed-immediate release bead, hydroxypropyl methylcellulose (HPMC) at about 1% to about 15% weight/weight of the delayed-immediate release bead, and talc at about 1% to about 10% weight/weight of the delayed-immediate release bead. In certain embodiments, the drug layer of the delayed-immediate release bead of pilocarpine, or pharmaceutically acceptable salt thereof, comprises pilocarpine hydrochloride at about 8.43% weight/weight of the delayed-immediate release bead, hydroxypropyl methylcellulose (HPMC) at about 1.69% weight/weight of the delayed-immediate release bead, and talc at about 2.53% weight/weight of the delayed-immediate release bead. In certain embodiments, the drug layer of the delayed-immediate release bead of pilocarpine, or pharmaceutically acceptable salt thereof, comprises pilocarpine hydrochloride at about 12.5% weight/weight of the delayed-immediate release bead, hydroxypropyl methylcellulose (HPMC) at about 12.5% weight/weight of the delayed-immediate release bead, and talc at about 6.25% weight/weight of the delayed-immediate release bead. In certain embodiments, the drug layer of the delayed-immediate release bead of pilocarpine, or pharmaceutically acceptable salt thereof, comprises pilocarpine hydrochloride at about 10.53% weight/weight of the delayed-immediate release bead, hydroxypropyl methylcellulose (HPMC) at about 10.53% weight/weight of the delayed-immediate release bead, and talc at about 5.26% weight/weight of the delayed-immediate release bead. In certain embodiments, the drug layer of the delayed-immediate release bead of pilocarpine, or pharmaceutically acceptable salt thereof, comprises pilocarpine hydrochloride at about 8.33% weight/weight of the delayed-immediate release bead, hydroxypropyl methylcellulose (HPMC) at about 8.33% weight/weight of the delayed-immediate release bead, and talc at about 4.17% weight/weight of the delayed-immediate release bead. In certain embodiments, the drug layer of the delayed-immediate release bead of pilocarpine, or pharmaceutically acceptable salt thereof, comprises pilocarpine hydrochloride at about 7.14% weight/weight of the delayed-immediate release bead, hydroxypropyl methylcellulose (HPMC) at about 7.14% weight/weight of the delayed-immediate release bead, and talc at about 3.57% weight/weight of the delayed-immediate release bead. In certain embodiments, the drug layer of the delayed-immediate release bead of pilocarpine, or pharmaceutically acceptable salt thereof, comprises pilocarpine hydrochloride at about 5.88% weight/weight of the delayed-immediate release bead, hydroxypropyl methylcellulose (HPMC) at about 5.88% weight/weight of the delayed-immediate release bead, and talc at about 2.94% weight/weight of the delayed-immediate release bead.

In certain embodiments, the delayed release coating of the delayed-immediate release bead of pilocarpine, or pharmaceutically acceptable salt thereof, comprises a pore former, a water insoluble polymer, and a plasticizer.

In certain embodiments, the ratio of pore former to water insoluble polymer is from 1:1 to 3:1 in the delayed release coating. In certain embodiments, the ratio of pore former to water insoluble polymer is from 1:1 to 1:3 in the delayed release coating. In certain embodiments, the ratio of pore former to water insoluble polymer is 1:3 in the delayed release coating.

In certain embodiments, the delayed release coating of the delayed-immediate release bead of pilocarpine, or pharmaceutically acceptable salt thereof, comprises the pore former at about 1% to about 35% weight/weight of the delayed-immediate release bead, the water insoluble polymer at about 5% to about 35% weight/weight of the delayed-immediate release bead, and the plasticizer at about 1% to about 10% weight/weight of the delayed-immediate release bead. In certain embodiments, the delayed release coating of the delayed-immediate release bead of pilocarpine, or pharmaceutically acceptable salt thereof, comprises the pore former at about 2.66% weight/weight of the delayed-immediate release bead, the water insoluble polymer at about 7.97% weight/weight of the delayed-immediate release bead, and the plasticizer at about 1.18% weight/weight of the delayed-immediate release bead. In certain embodiments, the delayed release coating of the delayed-immediate release bead of pilocarpine, or pharmaceutically acceptable salt thereof, comprises the pore former at about 17.08% weight/weight of the delayed-immediate release bead, the water insoluble polymer at about 17.08% weight/weight of the delayed-immediate release bead, and the plasticizer at about 3.33% weight/weight of the delayed-immediate release bead. In certain embodiments, the delayed release coating of the delayed-immediate release bead of pilocarpine, or pharmaceutically acceptable salt thereof, comprises the pore former at about 21.57% weight/weight of the delayed-immediate release bead, the water insoluble polymer at about 21.57% weight/weight of the delayed-immediate release bead, and the plasticizer at about 4.22% weight/weight of the delayed-immediate release bead. In certain embodiments, the delayed release coating of the delayed-immediate release bead of pilocarpine, or pharmaceutically acceptable salt thereof, comprises the pore former at about 26.56% weight/weight of the delayed-immediate release bead, the water insoluble polymer at about 26.56% weight/weight of the delayed-immediate release bead, and the plasticizer at about 5.22% weight/weight of the delayed-immediate release bead. In certain embodiments, the delayed release coating of the delayed-immediate release bead of pilocarpine, or pharmaceutically acceptable salt thereof, comprises the pore former at about 29.29% weight/weight of the delayed-immediate release bead, the water insoluble polymer at about 29.29% weight/weight of the delayed-immediate release bead, and the plasticizer at about 5.71% weight/weight of the delayed-immediate release bead. In certain embodiments, the delayed release coating of the delayed-immediate release bead of pilocarpine, or pharmaceutically acceptable salt thereof, comprises the pore former at about 32.16% weight/weight of the delayed-immediate release bead, the water insoluble polymer at about 32.16% weight/weight of the delayed-immediate release bead, and the plasticizer at about 6.27% weight/weight of the delayed-immediate release bead.

In certain embodiments, the pore former of the delayed release coating of the delayed-immediate release bead of pilocarpine, or pharmaceutically acceptable salt thereof, is a water soluble polymer. In certain embodiments, the pore former of the delayed release coating of the delayed-immediate release bead of pilocarpine, or pharmaceutically acceptable salt thereof, is selected from the group consisting of hydroxypropyl cellulose (HPC), polyethylene glycol (PEG), hydroxyl propyl methyl cellulose (HPMC), sucrose and other sugars, salts such as sodium bicarbonate, surfactants, hydroxypropyl methylcellulose (HPMC), polyvinylpyrrolidone (PVP), povidone K 29/32, lactose, mannitol, sucrose, liquid glucose, acacia, tragacanth, gelatin, starch paste, pregelantinized starch, alginic acid, cellulose, methyl cellulose, ethyl cellulose, hydroxyl propyl methyl cellulose (HPMC), hydroxyl propyl cellulose (HPC), sodium carboxy methyl cellulose, polyvinyl pyrrolidone (PVP), polyethylene glycol (PEG), polyvinyl alcohol, hydroxyethyl cellulose (HEC), polyacrylates, polyvinyl alcohol (PVA), polymethacrylates, EUDRAGIT®, Methylcellulose, cellulose acetate butyrates and combinations thereof.

In certain embodiments, the water insoluble polymer of the delayed release coating of the delayed-immediate release bead of pilocarpine, or pharmaceutically acceptable salt thereof, is selected from the group consisting of ethylcellulose (EC), polyacrylates, or polymethacrylates, cellulose acetate, waxes, shellac, and combinations thereof.

In certain embodiments, the plasticizer of the delayed release coating of the delayed-immediate release bead of pilocarpine, or pharmaceutically acceptable salt thereof, is selected from the group consisting of dibutyl sebacate, PEG400, low molecular weight PEG (MW<1500), glycerol, branched esters including triethyl citrate (TEC), triacetin, di-acid esters including dibutyl sebacate (DBS), diethyl phthalate (DEP), or fatty acids including fractionated coconut oil (FCO), Oleic acid (OA), hydrogenated vegetable oils, and combinations thereof.

In certain embodiments, the delayed release coating of the delayed-immediate release bead of pilocarpine, or pharmaceutically acceptable salt thereof, comprises hydroxypropyl cellulose (HPC) as the pore former, ethylcellulose (EC) as the water insoluble polymer, and dibutyl sebacate as the plasticizer. In certain embodiments, the ratio of HPC:EC is 1:3.

In certain embodiments, the delayed release coating of the delayed-immediate release bead of pilocarpine, or pharmaceutically acceptable salt thereof, comprises the hydroxypropyl cellulose (HPC) at about 1% to about 35% weight/weight of the delayed-immediate release bead, the ethylcellulose (EC) at about 5% to about 35% weight/weight of the delayed-immediate release bead, and the dibutyl sebacate at about 1% to about 10% weight/weight of the delayed-immediate release bead. In certain embodiments, the delayed release coating of the delayed-immediate release bead of pilocarpine, or pharmaceutically acceptable salt thereof, comprises the hydroxypropyl cellulose (HPC) at about 2.66% weight/weight of the delayed-immediate release bead, the ethylcellulose (EC) at about 7.97% weight/weight of the delayed-immediate release bead, and the dibutyl sebacate at about 1.18% weight/weight of the delayed-immediate release bead. In certain embodiments, the delayed release coating of the delayed-immediate release bead of pilocarpine, or pharmaceutically acceptable salt thereof, comprises the hydroxypropyl cellulose (HPC) at about 17.08% weight/weight of the delayed-immediate release bead, the ethylcellulose (EC) at about 17.08% weight/weight of the delayed-immediate release bead, and the dibutyl sebacate at about 3.33% weight/weight of the delayed-immediate release bead. In certain embodiments, the delayed release coating of the delayed-immediate release bead of pilocarpine, or pharmaceutically acceptable salt thereof, comprises the hydroxypropyl cellulose (HPC) at about 21.57% weight/weight of the delayed-immediate release bead, the ethylcellulose (EC) at about 21.57% weight/weight of the delayed-immediate release bead, and the dibutyl sebacate at about 4.22% weight/weight of the delayed-immediate release bead. In certain embodiments, the delayed release coating of the delayed-immediate release bead of pilocarpine, or pharmaceutically acceptable salt thereof, comprises the hydroxypropyl cellulose (HPC) at about 26.56% weight/weight of the delayed-immediate release bead, the ethylcellulose (EC) at about 26.56% weight/weight of the delayed-immediate release bead, and the dibutyl sebacate at about 5.22% weight/weight of the delayed-immediate release bead. In certain embodiments, the delayed release coating of the delayed-immediate release bead of pilocarpine, or pharmaceutically acceptable salt thereof, comprises the hydroxypropyl cellulose (HPC) at about 29.29% weight/weight of the delayed-immediate release bead, the ethylcellulose (EC) at about 29.29% weight/weight of the delayed-immediate release bead, and the dibutyl sebacate at about 5.71% weight/weight of the delayed-immediate release bead. In certain embodiments, the delayed release coating of the delayed-immediate release bead of pilocarpine, or pharmaceutically acceptable salt thereof, comprises the hydroxypropyl cellulose (HPC) at about 32.16% weight/weight of the delayed-immediate release bead, the ethylcellulose (EC) at about 32.16% weight/weight of the delayed-immediate release bead, and the dibutyl sebacate at about 6.27% weight/weight of the delayed-immediate release bead.

In certain embodiments, the drug layer or the delayed release coating of the delayed-immediate release bead of pilocarpine, or pharmaceutically acceptable salt thereof, further comprises an excipient selected from the group consisting of MCCPH102, Sodium startch glycolate, dicalcium phosphate, silicon dioxide, crospovidone XL 10, startch 1500, fast flo lactose, and combinations thereof.

In certain embodiments, each delayed-immediate release bead of pilocarpine, or pharmaceutically acceptable salt thereof, further comprises a seal coating coated on the delayed release coating of the delayed-immediate release bead. The seal coat provides the following advantageous properties to each bead: 1. Use of different color seal coats distinguishes the two different beads, 2. Enhanced stability by providing an additional layer of separation between the drug layer and the other beads (and other API) in the capsule, 3. Protects manufacturing operator by minimizing exposure to the APIs, and 4. Mitigates static electricity during the encapsulation process. In certain embodiments, the seal coating of the delayed-immediate release bead is at about 0.5% to about 5.0%. In certain embodiments, the seal coating of the immediate release bead is at about 1% to about 4.5%, about 1.5% to about 4.0%, about 2% to about 3.5%, about 2.5% to about 3.0%, about 1.0% to about 3%, about 1.0% to about 2.5%, or about 1.2% to about 2.0% weight/weight of the immediate release bead. In certain embodiments, the seal coating of the delayed-immediate release bead comprises about 3.85% weight/weight of the delayed-immediate release bead. In certain embodiments, the seal coating is a colored coat, such as any non-functional, commercially available coating, for example the Opadry® product line or their generic equivalents or shellac.

In some embodiments, each delayed-immediate release bead of pilocarpine, or pharmaceutically acceptable salt thereof, comprises 31.25% w/w 700 μm inert core, coated with a drug layer comprising 12.5% w/w pilocarpine chloride, 12.5% w/w hydroxypropyl methylcellulose (HPMC), and 6.25% w/w talc, a delayed release coating coated on the drug layer comprising 17.08% w/w hydroxypropyl cellulose, 17.08% w/w ethylcellulose, and 3.33% w/w dibutyl sebacate. In some embodiments, each delayed-immediate release bead of pilocarpine, or pharmaceutically acceptable salt thereof, comprises 18.75 mg/dose 700 μm inert core, coated with a drug layer comprising 7.5 mg/dose pilocarpine chloride, 7.5 mg/dose hydroxypropyl methylcellulose (HPMC), and 3.75 mg/dose talc, a delayed release coating coated on the drug layer comprising 10.25 mg/dose hydroxypropyl cellulose, 10.5 mg/dose ethylcellulose, and 2.0 mg/dose dibutyl sebacate. In embodiments described herein, the total fill per dose of a plurality of delayed-immediate release beads of pilocarpine, or pharmaceutically acceptable salt thereof, is 60 mg.

In some embodiments, each delayed-immediate release bead of pilocarpine, or pharmaceutically acceptable salt thereof, comprises 26.32% w/w 700 μm inert core, coated with a drug layer comprising 10.53% w/w pilocarpine chloride, 10.53% w/w hydroxypropyl methylcellulose (HPMC), and 5.26% w/w talc, a delayed release coating coated on the drug layer comprising 21.57% w/w hydroxypropyl cellulose, 21.57% w/w ethylcellulose, and 4.22% w/w dibutyl sebacate. In some embodiments, each delayed-immediate release bead of pilocarpine, or pharmaceutically acceptable salt thereof, comprises 18.75 mg/dose 700 μm inert core, coated with a drug layer comprising 7.5 mg/dose pilocarpine chloride, 7.5 mg/dose hydroxypropyl methylcellulose (HPMC), and 3.75 mg/dose talc, a delayed release coating coated on the drug layer comprising 15.37 mg/dose hydroxypropyl cellulose, 15.37 mg/dose ethylcellulose, and 3.01 mg/dose dibutyl sebacate. In embodiments described herein, the total fill per dose of a plurality of delayed-immediate release beads of pilocarpine, or pharmaceutically acceptable salt thereof, is 71.25 mg.

In some embodiments, each delayed-immediate release bead of pilocarpine, or pharmaceutically acceptable salt thereof, comprises 20.83% w/w 700 μm inert core, coated with a drug layer comprising 8.33% w/w pilocarpine chloride, 8.33% w/w hydroxypropyl methylcellulose (HPMC), and 4.17% w/w talc, a delayed release coating coated on the drug layer comprising 26.56% w/w hydroxypropyl cellulose, 26.56% w/w ethylcellulose, and 5.22% w/w dibutyl sebacate. In some embodiments, each delayed-immediate release bead of pilocarpine, or pharmaceutically acceptable salt thereof, comprises 18.75 mg/dose 700 μm inert core, coated with a drug layer comprising 7.5 mg/dose pilocarpine chloride, 7.5 mg/dose hydroxypropyl methylcellulose (HPMC), and 3.75 mg/dose talc, a delayed release coating coated on the drug layer comprising 23.9 mg/dose hydroxypropyl cellulose, 23.9 mg/dose ethylcellulose, and 4.7 mg/dose dibutyl sebacate. In embodiments described herein, the total fill per dose of a plurality of delayed-immediate release beads of pilocarpine, or pharmaceutically acceptable salt thereof, is 90 mg.

In some embodiments, each delayed-immediate release bead of pilocarpine, or pharmaceutically acceptable salt thereof, comprises 17.86% w/w 700 μm inert core, coated with a drug layer comprising 7.14% w/w pilocarpine chloride, 7.14% w/w hydroxypropyl methylcellulose (HPMC), and 3.57% w/w talc, a delayed release coating coated on the drug layer comprising 29.29% w/w hydroxypropyl cellulose, 29.29% w/w ethylcellulose, and 5.71% w/w dibutyl sebacate. In some embodiments, each delayed-immediate release bead of pilocarpine, or pharmaceutically acceptable salt thereof, comprises 18.75 mg/dose 700 μm inert core, coated with a drug layer comprising 7.5 mg/dose pilocarpine chloride, 7.5 mg/dose hydroxypropyl methylcellulose (HPMC), and 3.75 mg/dose talc, a delayed release coating coated on the drug layer comprising 30.75 mg/dose hydroxypropyl cellulose, 30.75 mg/dose ethylcellulose, and 6.0 mg/dose dibutyl sebacate. In embodiments described herein, the total fill per dose of a plurality of delayed-immediate release beads of pilocarpine, or pharmaceutically acceptable salt thereof, is 105 mg.

In some embodiments, each delayed-immediate release bead of pilocarpine, or pharmaceutically acceptable salt thereof, comprises 16.12% w/w 700 μm inert core, coated with a drug layer comprising 6.45% w/w pilocarpine chloride, 6.45% w/w hydroxypropyl methylcellulose (HPMC), and 3.22% w/w talc, a delayed release coating coated on the drug layer comprising 30.85% w/w hydroxypropyl cellulose, 30.85% w/w ethylcellulose, and 6.02% w/w dibutyl sebacate. In some embodiments, each delayed-immediate release bead of pilocarpine, or pharmaceutically acceptable salt thereof, comprises 18.75 mg/dose 700 μm inert core, coated with a drug layer comprising 7.5 mg/dose pilocarpine chloride, 7.5 mg/dose hydroxypropyl methylcellulose (HPMC), and 3.75 mg/dose talc, a delayed release coating coated on the drug layer comprising 35.88 mg/dose hydroxypropyl cellulose, 35.88 mg/dose ethylcellulose, and 7 mg/dose dibutyl sebacate. In embodiments described herein, the total fill per dose of a plurality of delayed-immediate release beads of pilocarpine, or pharmaceutically acceptable salt thereof, is 116.3 mg.

In some embodiments, the plurality of delayed-immediate release beads of pilocarpine, or a pharmaceutically acceptable salt thereof, is stable at 25° C./60% relative humidity (RH) for at least 4 weeks, at least 8 weeks, at least 12 weeks, at least 16 weeks, at least 20 weeks, or at least 24 weeks. In some embodiments, the plurality of delayed-immediate release beads of pilocarpine, or a pharmaceutically acceptable salt thereof, is stable at 25° C./60% relative humidity (RH) for at least 1 month, at least 3 months, at least 6 months, at least 9 months, at least 12 months, at least 18 months, or at least 24 months. In some embodiments, the plurality of delayed-immediate release beads of pilocarpine, or a pharmaceutically acceptable salt thereof, is stable at 30° C./75% RH for at least 4 weeks, at least 8 weeks, at least 12 weeks, at least 16 weeks, at least 20 weeks, or at least 24 weeks. In some embodiments, the plurality of delayed-immediate release beads of pilocarpine, or a pharmaceutically acceptable salt thereof, is stable at 40° C./75% RH for at least 4 weeks, at least 8 weeks, at least 12 weeks, at least 16 weeks, at least 20 weeks, or at least 24 weeks. In some embodiments, the plurality of delayed-immediate release beads of pilocarpine, or a pharmaceutically acceptable salt thereof, is stable at 40° C./75% RH for at least 1 month, at least 3 months, or at least 6 months. In some embodiments, the plurality of delayed-immediate release beads of pilocarpine, or a pharmaceutically acceptable salt thereof, is stable at 50° C./ambient conditions (AMB) for at least 4 weeks, at least 8 weeks, at least 12 weeks, at least 16 weeks, at least 20 weeks, or at least 24 weeks.

### Fixed Dose Compositions

Embodiments of the fixed dose pharmaceutical compositions comprising a 2-10 mg oxybutynin component and a 2-10 mg pilocarpine component, wherein the oxybutynin component is a plurality of immediate release beads and wherein the pilocarpine component is a plurality of delayed-immediate release beads may comprise any combination of the immediate release beads of oxybutynin described above and the delayed-immediate release beads of pilocarpine. Such embodiments also include specific compositions disclosed as follows:

In some embodiments, each delayed-immediate release bead of pilocarpine, or pharmaceutically acceptable salt thereof, comprises 71.69% w/w 500 μm inert core, coated with a drug layer comprising 8.43% w/w pilocarpine chloride, 1.69% w/w hydroxypropyl methylcellulose (HPMC), and 2.53% w/w talc, a delayed release coating coated on the drug layer comprising 2.66% w/w hydroxypropyl cellulose, 7.97% w/w ethylcellulose, and 1.18% w/w dibutyl sebacate, and 3.85% w/w of a seal coat coated on the delayed release coating. In some embodiments, each delayed-immediate release bead of pilocarpine, or pharmaceutically acceptable salt thereof, comprises 34 mg/dose 500 μm inert core, coated with a drug layer comprising 4.0 mg/dose pilocarpine chloride, 0.8 mg/dose hydroxypropyl methylcellulose (HPMC), and 1.2 mg/dose talc, a delayed release coating coated on the drug layer comprising 1.26 mg/dose hydroxypropyl cellulose, 3.78 mg/dose ethylcellulose, and 0.56 mg/dose dibutyl sebacate, and 1.82 mg/dose of a seal coat coated on the delayed release coating. In embodiments described herein, the total fill per dose of a plurality of delayed-immediate release beads of pilocarpine, or pharmaceutically acceptable salt thereof, is 47.42 mg.

In some embodiments, each delayed-immediate release bead of pilocarpine, or pharmaceutically acceptable salt thereof, comprises 71.69% w/w 500 μm inert core, coated with a drug layer comprising 8.43% w/w pilocarpine chloride, 1.69% w/w hydroxypropyl methylcellulose (HPMC), and 2.53% w/w talc, a delayed release coating coated on the drug layer comprising 2.66% w/w hydroxypropyl cellulose, 7.97% w/w ethylcellulose, and 1.18% w/w dibutyl sebacate, and 3.85% w/w of a seal coat coated on the delayed release coating. In some embodiments, each delayed-immediate release bead of pilocarpine, or pharmaceutically acceptable salt thereof, comprises 51 mg/dose 500 μm inert core, coated with a drug layer comprising 6.0 mg/dose pilocarpine chloride, 1.2 mg/dose hydroxypropyl methylcellulose (HPMC), and 1.8 mg/dose talc, a delayed release coating coated on the drug layer comprising 1.89 mg/dose hydroxypropyl cellulose, 5.67 mg/dose ethylcellulose, and 0.84 mg/dose dibutyl sebacate, and 2.76 mg/dose of a seal coat coated on the delayed release coating. In embodiments described herein, the total fill per dose of a plurality of delayed-immediate release beads of pilocarpine, or pharmaceutically acceptable salt thereof, is 71.13 mg.

In some embodiments, each delayed-immediate release bead of pilocarpine, or pharmaceutically acceptable salt thereof, comprises 71.69% w/w 500 μm inert core, coated with a drug layer comprising 8.43% w/w pilocarpine chloride, 1.69% w/w hydroxypropyl methylcellulose (HPMC), and 2.53% w/w talc, a delayed release coating coated on the drug layer comprising 2.66% w/w hydroxypropyl cellulose, 7.97% w/w ethylcellulose, and 1.18% w/w dibutyl sebacate, and 3.85% w/w of a seal coat coated on the delayed release coating. In some embodiments, each delayed-immediate release bead of pilocarpine, or pharmaceutically acceptable salt thereof, comprises 68 mg/dose 500 μm inert core, coated with a drug layer comprising 8.0 mg/dose pilocarpine chloride, 1.6 mg/dose hydroxypropyl methylcellulose (HPMC), and 2.4 mg/dose talc, a delayed release coating coated on the drug layer comprising 2.52 mg/dose hydroxypropyl cellulose, 7.56 mg/dose ethylcellulose, and 1.12 mg/dose dibutyl sebacate, and 3.65 mg/dose of a seal coat coated on the delayed release coating. In embodiments described herein, the total fill per dose of a plurality of delayed-immediate release beads of pilocarpine, or pharmaceutically acceptable salt thereof, is 94.85 mg.

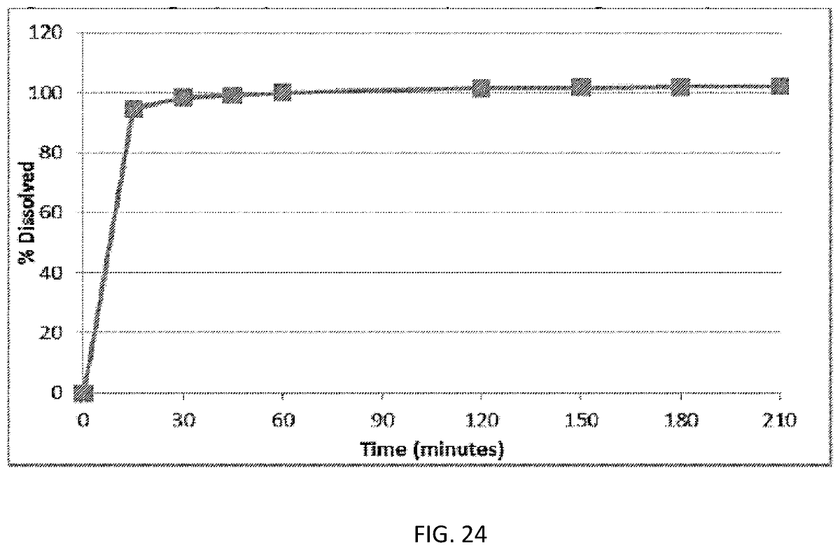

Embodiments described herein are directed to delayed-immediate release beads of pilocarpine, or pharmaceutically acceptable salt thereof, having a target dissolution profile which is equivalent to the Cₘₐₓ, Tₘₐₓ and AUC. The target dissolution profile is measured in vitro in 0.1N HCl as depicted in FIG. 24. In some embodiments, the AUC of the delayed-immediate release beads of pilocarpine, or pharmaceutically acceptable salt thereof, is about 67 ng*hr/ml to about 85 ng*hr/ml. In some embodiments, the Cₘₐₓ of the delayed-immediate release beads of pilocarpine, or pharmaceutically acceptable salt thereof, is about 23 ng/ml to about 30 ng/ml. In some embodiments, the Tₘₐₓ of the delayed-immediate release beads of pilocarpine, or pharmaceutically acceptable salt thereof, is about 1.75 hours to about 2 hours.

Embodiments described herein are directed to delayed-immediate release beads of pilocarpine, or pharmaceutically acceptable salt thereof, which achieve 80% to 125% of the target dissolution which is equivalent to the Cₘₐₓ, Tₘₐₓ and AUC achieved when pilocarpine is administered about 30 minutes after oxybutynin. In embodiments, the efficacy achieved and/or the side effect profile is better than what can be achieved by orally administering pilocarpine about 30 minutes after oxybutynin.

In some embodiments, the plurality of delayed-immediate release beads of pilocarpine, or a pharmaceutically acceptable salt thereof, release not more than 30% of pilocarpine, or a pharmaceutically acceptable salt thereof, within about 10 minutes or less of administration of the pharmaceutical composition to a subject and not less than 85% of pilocarpine, or a pharmaceutically acceptable salt thereof, after 45 minutes of administration of the pharmaceutical composition to a subject. In some embodiments, the plurality of delayed-immediate release beads of pilocarpine, or a pharmaceutically acceptable salt thereof, release less than about 30%, about 0% to about 25%, about 5% to about 20%, or about 10% to about 15% of pilocarpine, or a pharmaceutically acceptable salt thereof, within 10 minutes of administration of the pharmaceutical composition to a subject. In some embodiments, the plurality of delayed-immediate release beads of pilocarpine, or a pharmaceutically acceptable salt thereof, release about 85% or more, about 85% to about 100%, about 90% to about 100%, or about 95% to about 100% of pilocarpine, or a pharmaceutically acceptable salt thereof, about 45 minutes or more after administration of the pharmaceutical composition to a subject.

In embodiments described herein, the oxybutynin component as a plurality of immediate release beads and the pilocarpine component as a plurality of delayed-immediate release beads are encapsulated in a suitably sized capsule for the bead fill. Capsules can be HPMC or gelatin based hard capsules. Preferably, the capsules are of size 3 or smaller.

Embodiments of the present invention are directed to a fixed dose pharmaceutical composition comprising a capsule filled with 75 mg of a plurality of immediate release oxybutynin, or pharmaceutically acceptable salt thereof, beads as described herein and 60 mg of a plurality of delayed-immediate release pilocarpine, or pharmaceutically acceptable salt thereof, beads as described herein, wherein the fixed dose pharmaceutical composition delivers 7.5 mg of oxybutynin, or pharmaceutically acceptable salt thereof and 7.5 mg pilocarpine, or pharmaceutically acceptable salt thereof.

Embodiments of the present invention are directed to a fixed dose pharmaceutical composition comprising a capsule filled with 75 mg of a plurality of immediate release oxybutynin, or pharmaceutically acceptable salt thereof, beads as described herein and 71.25 mg of a plurality of delayed-immediate release pilocarpine, or pharmaceutically acceptable salt thereof, beads as described herein, wherein the fixed dose pharmaceutical composition delivers 7.5 mg of oxybutynin, or pharmaceutically acceptable salt thereof, and 7.5 mg pilocarpine, or pharmaceutically acceptable salt thereof.

Embodiments of the present invention are directed to a fixed dose pharmaceutical composition comprising a capsule filled with 75 mg of a plurality of immediate release oxybutynin, or pharmaceutically acceptable salt thereof, beads as described herein and 90 mg of a plurality of delayed-immediate release pilocarpine, or pharmaceutically acceptable salt thereof, beads as described herein, wherein the fixed dose pharmaceutical composition delivers 7.5 mg of oxybutynin, or pharmaceutically acceptable salt thereof, and 7.5 mg pilocarpine, or pharmaceutically acceptable salt thereof.

Embodiments of the present invention are directed to a fixed dose pharmaceutical composition comprising a capsule filled with 75 mg of a plurality of immediate release oxybutynin, or pharmaceutically acceptable salt thereof, beads as described herein and 105 mg of a plurality of delayed-immediate release pilocarpine, or pharmaceutically acceptable salt thereof, beads as described herein, wherein the fixed dose pharmaceutical composition delivers 7.5 mg of oxybutynin, or pharmaceutically acceptable salt thereof, and 7.5 mg pilocarpine, or pharmaceutically acceptable salt thereof.

Embodiments of the present invention are directed to a fixed dose pharmaceutical composition comprising a capsule filled with 75 mg of a plurality of immediate release oxybutynin, or pharmaceutically acceptable salt thereof, beads as described herein and 127.5 mg of a plurality of delayed-immediate release pilocarpine, or pharmaceutically acceptable salt thereof, beads as described herein, wherein the fixed dose pharmaceutical composition delivers 7.5 mg of oxybutynin, or pharmaceutically acceptable salt thereof, and 7.5 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 47.42 mg of a plurality of delayed-immediate release pilocarpine, or pharmaceutically acceptable salt thereof, beads as described herein, wherein the fixed dose pharmaceutical composition delivers 2 mg of oxybutynin, or pharmaceutically acceptable salt thereof, and 4 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 71.13 mg of a plurality of delayed-immediate release pilocarpine, or pharmaceutically acceptable salt thereof, beads as described herein, wherein the fixed dose pharmaceutical composition delivers 2 mg of oxybutynin, or pharmaceutically acceptable salt thereof, and 6 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 94.85 mg of a plurality of delayed-immediate release pilocarpine, or pharmaceutically acceptable salt thereof, beads as described herein, wherein the fixed dose pharmaceutical composition delivers 2 mg of oxybutynin, or pharmaceutically acceptable salt thereof, and 8 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 47.42 mg of a plurality of delayed-immediate release pilocarpine, or pharmaceutically acceptable salt thereof, beads as described herein, wherein the fixed dose pharmaceutical composition delivers 4 mg of oxybutynin, or pharmaceutically acceptable salt thereof, and 4 mg pilocarpine, or pharmaceutically acceptable salt thereof. Embodiments of the present invention are directed to a fixed dose pharmaceutical composition comprising a capsule filled with 40.8 mg of a plurality of immediate release oxybutynin, or pharmaceutically acceptable salt thereof, beads as described herein and 47.42 mg of a plurality of delayed-immediate release pilocarpine, or pharmaceutically acceptable salt thereof, beads as described herein, wherein the fixed dose pharmaceutical composition delivers 4 mg of oxybutynin, or pharmaceutically acceptable salt thereof, and 4 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 71.13 mg of a plurality of delayed-immediate release pilocarpine, or pharmaceutically acceptable salt thereof, beads as described herein, wherein the fixed dose pharmaceutical composition delivers 4 mg of oxybutynin, or pharmaceutically acceptable salt thereof, and 6 mg pilocarpine, or pharmaceutically acceptable salt thereof. Embodiments of the present invention are directed to a fixed dose pharmaceutical composition comprising a capsule filled with 40.8 mg of a plurality of immediate release oxybutynin, or pharmaceutically acceptable salt thereof, beads as described herein and 71.13 mg of a plurality of delayed-immediate release pilocarpine, or pharmaceutically acceptable salt thereof, beads as described herein, wherein the fixed dose pharmaceutical composition delivers 4 mg of oxybutynin, or pharmaceutically acceptable salt thereof, and 6 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 94.85 mg of a plurality of delayed-immediate release pilocarpine, or pharmaceutically acceptable salt thereof, beads as described herein, wherein the fixed dose pharmaceutical composition delivers 4 mg of oxybutynin, or pharmaceutically acceptable salt thereof, and 8 mg pilocarpine, or pharmaceutically acceptable salt thereof. Embodiments of the present invention are directed to a fixed dose pharmaceutical composition comprising a capsule filled with 40.8 mg of a plurality of immediate release oxybutynin, or pharmaceutically acceptable salt thereof, beads as described herein and 94.85 mg of a plurality of delayed-immediate release pilocarpine, or pharmaceutically acceptable salt thereof, beads as described herein, wherein the fixed dose pharmaceutical composition delivers 4 mg of oxybutynin, or pharmaceutically acceptable salt thereof, and 8 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 47.42 mg of a plurality of delayed-immediate release pilocarpine, or pharmaceutically acceptable salt thereof, beads as described herein, wherein the fixed dose pharmaceutical composition delivers 6 mg of oxybutynin, or pharmaceutically acceptable salt thereof, and 4 mg pilocarpine, or pharmaceutically acceptable salt thereof. Embodiments of the present invention are directed to a fixed dose pharmaceutical composition comprising a capsule filled with 61.2 mg of a plurality of immediate release oxybutynin, or pharmaceutically acceptable salt thereof, beads as described herein and 47.42 mg of a plurality of delayed-immediate release pilocarpine, or pharmaceutically acceptable salt thereof, beads as described herein, wherein the fixed dose pharmaceutical composition delivers 6 mg of oxybutynin, or pharmaceutically acceptable salt thereof, and 4 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 71.13 mg of a plurality of delayed-immediate release pilocarpine, or pharmaceutically acceptable salt thereof, beads as described herein, wherein the fixed dose pharmaceutical composition delivers 6 mg of oxybutynin, or pharmaceutically acceptable salt thereof, and 6 mg pilocarpine, or pharmaceutically acceptable salt thereof. Embodiments of the present invention are directed to a fixed dose pharmaceutical composition comprising a capsule filled with 61.2 mg of a plurality of immediate release oxybutynin, or pharmaceutically acceptable salt thereof, beads as described herein and 71.13 mg of a plurality of delayed-immediate release pilocarpine, or pharmaceutically acceptable salt thereof, beads as described herein, wherein the fixed dose pharmaceutical composition delivers 6 mg of oxybutynin, or pharmaceutically acceptable salt thereof, and 6 mg pilocarpine, or pharmaceutically acceptable salt thereof.

Embodiments of the present invention are directed to a fixed dose pharmaceutical composition comprising a capsule filled with 80 mg of a plurality of immediate release oxybutynin, or pharmaceutically acceptable salt thereof, beads as described herein and 94.85 mg of a plurality of delayed-immediate release pilocarpine, or pharmaceutically acceptable salt thereof, beads as described herein, wherein the fixed dose pharmaceutical composition delivers 6 mg of oxybutynin, or pharmaceutically acceptable salt thereof, and 8 mg pilocarpine, or pharmaceutically acceptable salt thereof. Embodiments of the present invention are directed to a fixed dose pharmaceutical composition comprising a capsule filled with 61.2 mg of a plurality of immediate release oxybutynin, or pharmaceutically acceptable salt thereof, beads as described herein and 94.85 mg of a plurality of delayed-immediate release pilocarpine, or pharmaceutically acceptable salt thereof, beads as described herein, wherein the fixed dose pharmaceutical composition delivers 6 mg of oxybutynin, or pharmaceutically acceptable salt thereof, and 8 mg pilocarpine, or pharmaceutically acceptable salt thereof.

Embodiments of the present invention are directed to a fixed dose pharmaceutical composition comprising a capsule filled with 81.6 mg of a plurality of immediate release oxybutynin, or pharmaceutically acceptable salt thereof, beads as described herein and 47.42 mg of a plurality of delayed-immediate release pilocarpine, or pharmaceutically acceptable salt thereof, beads as described herein, wherein the fixed dose pharmaceutical composition delivers 8 mg of oxybutynin, or pharmaceutically acceptable salt thereof, and 4 mg pilocarpine, or pharmaceutically acceptable salt thereof.

Embodiments of the present invention are directed to a fixed dose pharmaceutical composition comprising a capsule filled with 81.6 mg of a plurality of immediate release oxybutynin, or pharmaceutically acceptable salt thereof, beads as described herein and 71.13 mg of a plurality of delayed-immediate release pilocarpine, or pharmaceutically acceptable salt thereof, beads as described herein, wherein the fixed dose pharmaceutical composition delivers 8 mg of oxybutynin, or pharmaceutically acceptable salt thereof, and 6 mg pilocarpine, or pharmaceutically acceptable salt thereof.

Embodiments of the present invention are directed to a fixed dose pharmaceutical composition comprising a capsule filled with 81.6 mg of a plurality of immediate release oxybutynin, or pharmaceutically acceptable salt thereof, beads as described herein and 94.85 mg of a plurality of delayed-immediate release pilocarpine, or pharmaceutically acceptable salt thereof, beads as described herein, wherein the fixed dose pharmaceutical composition delivers 8 mg of oxybutynin, or pharmaceutically acceptable salt thereof, and 8 mg pilocarpine, or pharmaceutically acceptable salt thereof.

In embodiments, the plurality of immediate release beads of oxybutynin and the plurality of delayed-immediate release beads of pilocarpine of the fixed dose pharmaceutical compositions are as described above.

In certain embodiments, the fixed dose pharmaceutical composition comprises a plurality of immediate release beads of oxybutynin, or pharmaceutically acceptable salt thereof, providing a total amount of 8 mg of oxybutynin and a plurality of delayed-immediate release beads of pilocarpine, or pharmaceutically acceptable salt thereof, providing a total amount of 8 mg of pilocarpine.

In certain embodiments, the fixed dose pharmaceutical composition comprises a plurality of immediate release beads of oxybutynin, or pharmaceutically acceptable salt thereof, providing a total amount of 6 mg of oxybutynin and a plurality of delayed-immediate release beads of pilocarpine, or pharmaceutically acceptable salt thereof, providing a total amount of 6 mg of pilocarpine.

In certain embodiments, the fixed dose pharmaceutical composition comprises a plurality of immediate release beads of oxybutynin, or pharmaceutically acceptable salt thereof, providing a total amount of 4 mg of oxybutynin and a plurality of delayed-immediate release beads of pilocarpine, or pharmaceutically acceptable salt thereof, providing a total amount of 4 mg of pilocarpine.

In certain embodiments, the fixed dose pharmaceutical composition comprises a plurality of immediate release beads of oxybutynin, or pharmaceutically acceptable salt thereof, providing a total amount of 8 mg of oxybutynin and a plurality of delayed-immediate release beads of pilocarpine, or pharmaceutically acceptable salt thereof, providing a total amount of 6 mg of pilocarpine.

In certain embodiments, the fixed dose pharmaceutical composition comprises a plurality of immediate release beads of oxybutynin, or pharmaceutically acceptable salt thereof, providing a total amount of 8 mg of oxybutynin and a plurality of delayed-immediate release beads of pilocarpine, or pharmaceutically acceptable salt thereof, providing a total amount of 4 mg of pilocarpine.

In certain embodiments, the fixed dose pharmaceutical composition comprises a plurality of immediate release beads of oxybutynin, or pharmaceutically acceptable salt thereof, providing a total amount of 6 mg of oxybutynin and a plurality of delayed-immediate release beads of pilocarpine, or pharmaceutically acceptable salt thereof, providing a total amount of 8 mg of pilocarpine.

In certain embodiments, the fixed dose pharmaceutical composition comprises a plurality of immediate release beads of oxybutynin, or pharmaceutically acceptable salt thereof, providing a total amount of 6 mg of oxybutynin and a plurality of delayed-immediate release beads of pilocarpine, or pharmaceutically acceptable salt thereof, providing a total amount of 4 mg of pilocarpine.

In certain embodiments, the fixed dose pharmaceutical composition comprises a plurality of immediate release beads of oxybutynin, or pharmaceutically acceptable salt thereof, providing a total amount of 4 mg of oxybutynin and a plurality of delayed-immediate release beads of pilocarpine, or pharmaceutically acceptable salt thereof, providing a total amount of 8 mg of pilocarpine.

In certain embodiments, the fixed dose pharmaceutical composition comprises a plurality of immediate release beads of oxybutynin, or pharmaceutically acceptable salt thereof, providing a total amount of 4 mg of oxybutynin and a plurality of delayed-immediate release beads of pilocarpine, or pharmaceutically acceptable salt thereof, providing a total amount of 6 mg of pilocarpine.

In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 25° C./60% relative humidity (RH) for at least 4 weeks, at least 8 weeks, at least 12 weeks, at least 16 weeks, at least 20 weeks, or at least 24 weeks. In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 25° C./60% relative humidity (RH) for at least 1 month, at least 3 months, at least 6 months, at least 9 months, at least 12 months, at least 18 months, or at least 24 months. In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 30° C./75% RH for at least 4 weeks, at least 8 weeks, at least 12 weeks, at least 16 weeks, at least 20 weeks, or at least 24 weeks. In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 30° C./75% RH for at least 1 month, at least 3 months, at least 6 months, at least 9 months, or at least 12 months. In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 40° C./75% RH for at least 4 weeks, at least 8 weeks, at least 12 weeks, at least 16 weeks, at least 20 weeks, or at least 24 weeks. In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 40° C./75% RH for at least 1 month, at least 3 months, or at least 6 months. In some embodiments, the fixed dose pharmaceutical compositions described herein comprising a plurality of immediate release beads of oxybutynin, or pharmaceutically acceptable salt thereof, and a plurality of delayed-immediate release beads of pilocarpine, or pharmaceutically acceptable salt thereof, are stable at 50° C./ambient conditions (AMB) for at least 4 weeks, at least 8 weeks, at least 12 weeks, at least 16 weeks, at least 20 weeks, or at least 24 weeks.

The pharmaceutical composition can also contain components conventional in pharmaceutical preparations, e.g. one or more carriers, binders, lubricants, excipients (e.g., to impart controlled release characteristics), pH modifiers, sweeteners, bulking agents, coloring agents or further active agents.

In certain embodiments, pharmaceutical compositions of the invention may also include an antimicrobial agent for preventing or deterring microbial growth, such as for example benzalkonium chloride, benzethonium chloride, benzyl alcohol, cetylpyridinium chloride, chlorobutanol, phenol, phenylethyl alcohol, phenylmercuric nitrate, thimersol, and any combinations thereof.

One or more antioxidants may also be employed. Antioxidants, which can reduce or prevent oxidation and thus deterioration of the pharmaceutical composition, may include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and any combinations thereof.

One or more surfactants may also be included in pharmaceutical compositions of the invention. For example, suitable surfactants may include, but are not limited to polysorbates, such as Tween 20 and Tween 80, and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other cations.

Acids or bases may also be present in pharmaceutical compositions of the invention. For example, acids may include but are not limited to hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and any combinations thereof. Examples of bases include, but are not limited to sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and any combinations thereof.

The amount of any individual excipient in the oral dosage pharmaceutical composition will vary depending on the nature and function of the excipient, oral dosage delivery vehicle and particular needs of the pharmaceutical composition. In some instances, the optimal amount of any individual excipient is determined through routine experimentation, i.e., by preparing pharmaceutical compositions containing varying amounts of the excipient (ranging from low to high), examining the stability and other parameters, and then determining the range at which optimal performance is attained with no significant adverse effects. Generally, however, the excipient(s) will be present in the oral dosage pharmaceutical composition in an amount of about 1% to about 99% by weight, such as from about 5% to about 98% by weight, such as from about 15 to about 95% by weight of the excipient, including less than 30% by weight. Pharmaceutical excipients along with other excipients that may be employed in pharmaceutical compositions of interest are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000, the disclosure of which is herein incorporated by reference.

Where the pharmaceutical compositions are oral formulations, the pharmaceutical composition may include appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Methods for Treating Hyperhidrosis

Embodiments are directed to methods of treating hyperhidrosis comprising administering to a subject in need thereof a fixed dose pharmaceutical composition as described herein.

Embodiments are directed to methods of treating hyperhidrosis comprising administering to a subject in need thereof a fixed dose pharmaceutical composition comprising a combination of a plurality of immediate release beads of oxybutynin, or pharmaceutically acceptable salt thereof, and a plurality of delayed-immediate release beads of pilocarpine, or pharmaceutically acceptable salt thereof.

In embodiments, the fixed dose pharmaceutical compositions are administered orally to the subject in need thereof. In embodiments, the fixed dose pharmaceutical compositions are administered as needed, once a day, twice a day or three times a day. In preferred embodiments, the fixed dose pharmaceutical compositions is administered twice a day.

In embodiments, the hyperhidrosis is selected from the group consisting of primary (focal) hyperhidrosis, secondary hyperhidrosis, axillary hyperhidrosis, palmar hyperhidrosis, plantar hyperhidrosis, craniofacial hyperhidrosis, generalized hyperhidrosis, or compensatory sweating post-surgery.

In embodiments, treating hyperhidrosis includes reducing excessive sweating experienced by the subject, such as where excessive sweating by the subject is reduced by about 5% or more, by 10% or more, by 20% or more, by 25% or more, by 50% or more, by 75% or more, by 90% or more, or by 99% or more as reported by the subject or as determined by gravimetric assessments, on the Hyperhidrosis Disease Severity Scale (HDSS), by measurement by transdermal epidural water vapor loss (e.g., Vapometer, Delfin Technologies, Kuopio Finland), on the Hyperhidrosis Visual Quantification Scale (HHVQS, e.g., HHVQSa or HHVQSp), on the Hyperhidrosis Visual Analog Scale (HHVAS) or any combination thereof. In other embodiments, treating hyperhidrosis includes eliminating excessive sweating experienced by the subject.

In some embodiments, the treatment is sufficient to reduce excessive sweating by about 5% to about 25%, about 25% to about 50%, about 75% to about 90%, or by about 90% to about 99%, as reported by the subject or as determined by gravimetric assessments, on the Hyperhidrosis Disease Severity Scale (HDSS), by measurement by transdermal epidural water vapor loss (e.g., Vapometer, Delfin Technologies, Kuopio Finland), on the Hyperhidrosis Visual Quantification Scale (HHVQS, e.g., HHVQSa or HHVQSp), on the Hyperhidrosis Visual Analog Scale (HHVAS) or any combination thereof.

In embodiments, pilocarpine, or pharmaceutically acceptable salt thereof, is reduces one or more side effects caused by oxybutynin, or a pharmaceutically acceptable salt thereof, such as dry mouth. In certain embodiments, the side effect of dry mouth is reduced by about 25% or more, about 50% or more, about 75% or more, about 90% or more, about 95% or more, or about 99% or more as reported by the subject or as assessed by the Dry Mouth Visual Analog Scale (DMVAS), by measuring salivary flow, by a Dry Mouth Severity/Incidence Questionnaire, e.g., as described herein, or a combination thereof. In some embodiments, the severity of dry mouth is reduced by about 25% to about 50%, about 50% to about 75%, about 75% to about 90%, about 90% to about 95%, or about 95% to about 99% as reported by the subject or as assessed by the Dry Mouth Visual Analog Scale (DMVAS), by measuring salivary flow, by a Dry Mouth Severity/Incidence Questionnaire, e.g., as described herein, or a combination thereof. In embodiments, the dry mouth caused by oxybutynin, or pharmaceutically acceptable salt thereof, is completely alleviated as reported by the subject or as assessed by the Dry Mouth Visual Analog Scale (DMVAS), by measuring salivary flow, by a Dry Mouth Severity/Incidence Questionnaire, e.g., as described herein, or a combination thereof.

In some embodiments, pilocarpine, or pharmaceutically acceptable salt thereof, is administered with oxybutynin, or a pharmaceutically acceptable salt thereof, to the subject in an amount sufficient to reduce the number of incidences of moderate to severe dry mouth by about 25% to about 50%, about 50% to about 75%, about 75% to about 90%, about 90% to about 95%, or about 95% to about 99% as reported by the subject or as assessed by the Dry Mouth Visual Analog Scale (DMVAS), by measuring salivary flow, by a Dry Mouth Severity/Incidence Questionnaire, e.g., as described herein, or a combination thereof.

In some embodiments, pilocarpine, or pharmaceutically acceptable salt thereof, is administered with oxybutynin, or a pharmaceutically acceptable salt thereof, to the subject in an amount sufficient to completely eliminate the number of incidences of moderate or severe dry mouth caused by oxybutynin as reported by the subject or as assessed by the Dry Mouth Visual Analog Scale (DMVAS), by measuring salivary flow, by a Dry Mouth Severity/Incidence Questionnaire, e.g., as described herein, or a combination thereof.

Embodiments described herein are directed to methods of treating hyperhidrosis comprising administering to a subject in need thereof a fixed dose pharmaceutical composition of oxybutynin, or pharmaceutically acceptable salt thereof, and pilocarpine, or pharmaceutically acceptable salt thereof, as described herein wherein the oral administration of the fixed dose pharmaceutical composition reaches a target PK profile. As used herein, a “PK profile” refers to a profile of drug concentration in blood or plasma. Such a profile can be a relationship of drug concentration over time (i.e., a “concentration-time PK profile”) or a relationship of drug concentration versus number of doses administered (i.e., a “concentration-dose PK profile”.) A PK profile is characterized by PK parameters. As used herein, a “PK parameter” refers to a measure of drug concentration in blood or plasma, such as: 1) “drug Cₘₐₓ”, the maximum concentration of drug achieved in blood or plasma; 2) “drug Tₘₐₓ”, the time elapsed following ingestion to achieve Cₘₐₓ; and 3) “drug exposure”, the total concentration of drug present in blood or plasma over a selected period of time, which can be measured using the area under the curve (AUC) of a time course of drug release over a selected period of time (t). Modification of one or more PK parameters provides for a modified PK profile.

Embodiments described herein are directed to methods of treating hyperhidrosis comprising orally administering to a subject in need thereof a fixed dose pharmaceutical composition of oxybutynin, or pharmaceutically acceptable salt thereof, and pilocarpine, or pharmaceutically acceptable salt thereof, as described herein wherein the oral administration of the fixed dose pharmaceutical composition reaches a target Cₘₐₓ of about 3 ng/ml to about 6 ng/ml for oxybutynin, or pharmaceutically acceptable salt thereof, and about 23 ng/ml to about 30 ng/ml for pilocarpine, or pharmaceutically acceptable salt thereof.

Embodiments described herein are directed to methods of treating hyperhidrosis comprising orally administering to a subject in need thereof a fixed dose pharmaceutical composition of oxybutynin, or pharmaceutically acceptable salt thereof, and pilocarpine, or pharmaceutically acceptable salt thereof, as described herein wherein the oral administration of the fixed dose pharmaceutical composition reaches a target Tₘₐₓ of about 0.75 hour to about 1 hour for oxybutynin, or pharmaceutically acceptable salt thereof, and about 1.75 hours to about 2 hours for pilocarpine, or pharmaceutically acceptable salt thereof.

Embodiments described herein are directed to methods of treating hyperhidrosis comprising orally administering to a subject in need thereof a fixed dose pharmaceutical composition of oxybutynin, or pharmaceutically acceptable salt thereof, and pilocarpine, or pharmaceutically acceptable salt thereof, as described herein wherein the oral administration of the fixed dose pharmaceutical composition reaches a target AUC of about 8 ng*hr/ml to about 12 ng*hr/ml for oxybutynin, or pharmaceutically acceptable salt thereof, and about 67 ng*hr/ml to about 85 ng*hr/ml for pilocarpine, or pharmaceutically acceptable salt thereof.

Embodiments described herein are directed to methods of treating hyperhidrosis comprising orally administering to a subject in need thereof a fixed dose pharmaceutical composition of oxybutynin, or pharmaceutically acceptable salt thereof, and pilocarpine, or pharmaceutically acceptable salt thereof, as described herein wherein the oral administration of the fixed pharmaceutical composition achieves the 80% to 125% of the target dissolution which is equivalent to the Cₘₐₓ, Tₘₐₓ and AUC achieved when pilocarpine is administered about 30 minutes after oxybutynin.

In the foregoing embodiments, the efficacy achieved and/or the side effect profile is better than what can be achieved by orally administering pilocarpine about 30 minutes after oxybutynin.

Embodiments described herein are directed to methods of treating hyperhidrosis comprising orally administering to a subject in need thereof a fixed dose pharmaceutical composition comprising an oxybutynin component and a pilocarpine component, wherein the oral administration of the fixed dose pharmaceutical composition releases not more than 30% of pilocarpine, or pharmaceutically acceptable salt thereof, within 10 minutes after administration and not less than 85% after 45 minutes after administration, and greater than 90% of oxybutynin, or pharmaceutically acceptable salt thereof, within 10 minutes after administration.

Aspects, Aspects, including embodiments, of the subject matter described herein may be beneficial alone or in combination, with one or more other aspects or embodiments. Without limiting the description, certain non-limiting aspects of the disclosure numbered 1-40 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A fixed dose pharmaceutical composition comprising an oxybutynin component and a pilocarpine component,
wherein the oxybutynin component is a plurality of immediate release beads, each immediate release bead comprises:
   an inert core of the immediate release bead having a diameter of about 450 μm to about 700 μm, and
   a drug layer of the immediate release bead comprising oxybutynin, or a pharmaceutically acceptable salt thereof, coated on the inert core of the immediate release bead, and
wherein the pilocarpine component is a plurality of delayed-immediate release beads, each delayed-immediate release bead comprises:
   an inert core of the delayed-immediate release bead having a diameter of about 250 μm to about 700 μm,
   a drug layer of the delayed-immediate release bead comprising pilocarpine, or a pharmaceutically acceptable salt thereof, coated on the inert core of the delayed-immediate release bead, and
   a delayed release coating of the delayed-immediate release bead coated on the drug layer of the delayed-immediate release bead,
wherein the oxybutynin component contains about 2 mg to about 10 mg of oxybutynin, or pharmaceutically acceptable salt thereof, and
wherein the pilocarpine component contains about 2 mg to about 10 mg of pilocarpine, or pharmaceutically acceptable salt thereof.

2. The fixed dose pharmaceutical composition of 1, wherein the plurality of immediate release beads and the plurality of delayed-immediate release beads are encapsulated in a size 3 capsule.

3. The fixed dose pharmaceutical composition of 1, wherein each of the immediate release beads further comprises a seal coating coated on the drug layer of the immediate release bead.

4. The fixed dose pharmaceutical composition of 3, wherein the seal coating of the immediate release bead is at about 0.5% to about 5.0% weight/weight of the immediate release bead.

5. The fixed dose pharmaceutical composition of 1, wherein the inert core of the immediate release bead is a microcrystalline cellulose sphere at about 70% to about 95% weight/weight of the immediate release bead.

6. The fixed dose pharmaceutical composition of 1, wherein the drug layer of the immediate release bead further comprises a binder, and an anti-tacking agent.

7. The fixed dose pharmaceutical composition of 6, wherein the oxybutynin, or pharmaceutically acceptable salt thereof, is about 2% to about 15% weight/weight of the immediate release bead, the binder is about 2% to about 15% weight/weight of the immediate release bead, and the anti-tacking agent is about 1% to about 10% weight/weight of the immediate release bead.

8. The fixed dose pharmaceutical composition of 6, wherein the oxybutynin, or pharmaceutically acceptable salt thereof, to binder ratio is about 1:1.

9. The fixed dose pharmaceutical composition of 6, wherein the binder is hydroxypropyl methylcellulose (HPMC) and the anti-tacking agent is talc.

10. The fixed dose pharmaceutical composition of 9, wherein oxybutynin chloride is at about 9.8% weight/weight of the immediate release bead, hydroxypropyl methylcellulose (HPMC) is at about 9.8% weight/weight of the immediate release bead, and talc is at about 4.9% weight/weight of the immediate release bead.

11. The fixed dose pharmaceutical composition of 1, wherein the plurality of immediate release beads of oxybutynin, or a pharmaceutically acceptable salt thereof, release about 90% or more, about 95% or more, about 99% or more, or 100% of oxybutynin, or a pharmaceutically acceptable salt thereof, within about 10 minutes or less of administration of the fixed dose pharmaceutical composition to a subject.

12. The fixed dose pharmaceutical composition of 1, wherein the inert core of the delayed-immediate release bead is a microcrystalline cellulose sphere at about 10% to about 75% weight/weight of the delayed-immediate release bead.

13. The fixed dose pharmaceutical composition of 1, wherein the drug layer of the delayed-immediate release bead further comprises a binder and an anti-tacking agent.

14. The fixed dose pharmaceutical composition of 13, wherein the drug layer of the delayed-immediate release bead comprises pilocarpine, or pharmaceutically acceptable salt thereof, at about 5% to about 15% weight/weight of the delayed-immediate release bead, the binder at about 1% to about 15% weight/weight of the delayed-immediate release bead, and the anti-tacking agent at about 1% to about 10% weight/weight of the delayed-immediate release bead.

15. The fixed dose pharmaceutical composition of 13, wherein the pilocarpine, or pharmaceutically acceptable salt thereof, to binder ratio is about 5:1.

16. The fixed dose pharmaceutical composition of 13, wherein the drug layer comprises pilocarpine hydrochloride, hydroxypropyl methylcellulose (HPMC) as the binder, and talc as the anti-tacking agent.

17. The fixed dose pharmaceutical composition of claim 16, wherein pilocarpine hydrochloride is at about 8.43% weight/weight of the delayed-immediate release bead, hydroxypropyl methylcellulose (HPMC) is at about 1.69% weight/weight of the delayed-immediate release bead, and talc is at about 2.53% weight/weight of the delayed-immediate release bead.

18. The fixed dose pharmaceutical composition of 1, wherein the delayed release coating of the delayed-immediate release bead comprises a pore former, a water insoluble polymer, and a plasticizer.

19. The fixed dose pharmaceutical composition of 18, wherein the pore former is about 1% to about 35% weight/weight of the delayed-immediate release bead, the water insoluble polymer is about 5% to about 35% weight/weight of the delayed-immediate release bead, and the plasticizer is about 1% to about 10% weight/weight of the delayed-immediate release bead.

20. The fixed dose pharmaceutical composition of 18, wherein the pore former to water insoluble polymer ratio is about 1:3.

21. The fixed dose pharmaceutical composition of 18, wherein the delayed release coating comprises hydroxypropyl cellulose (HPC) as the pore former, ethylcellulose (EC) as the water insoluble polymer, and dibutyl sebacate as the plasticizer.

22. The fixed dose pharmaceutical composition of 21, wherein the delayed release coating comprises hydroxypropyl cellulose (HPC) at 2.66% weight/weight of the delayed-immediate release bead, ethylcellulose (EC) at 7.97% weight/weight of the delayed-immediate release bead, and dibutyl sebacate at 1.18% weight/weight of the delayed-immediate release bead.

23. The fixed dose pharmaceutical composition of 1, wherein each of the delayed-immediate release beads further comprises a seal coating coated on the delayed release coating of the delayed-immediate release bead.

24. The fixed dose pharmaceutical composition of 23, wherein the seal coating of the delayed-immediate release bead is at about 0.5% to about 5.0% weight/weight of the delayed-immediate release bead.

25. The fixed dose pharmaceutical composition of 1, wherein the plurality of delayed-immediate release beads of pilocarpine, or a pharmaceutically acceptable salt thereof, release not more than 30% of pilocarpine, or a pharmaceutically acceptable salt thereof, in 10 minutes and not less than 85% of pilocarpine, or a pharmaceutically acceptable salt thereof, at 45 minutes of administration of the pharmaceutical composition to a subject.

26. A pharmaceutical composition comprising:
a plurality of immediate release beads, each bead comprises:
   an inert core of the immediate release bead having a diameter of about 450 μm to about 700 μm, and
   a drug layer of the immediate release bead comprising oxybutynin, or a pharmaceutically acceptable salt thereof, coated on the inert core of the immediate release bead,
   wherein the plurality of immediate release beads contains a total amount of about 2 mg to about 10 mg of oxybutynin, or pharmaceutically acceptable salt thereof.

27. The pharmaceutical composition of 26, wherein the drug layer of the immediate release bead is further coated with a seal coat.

28. The pharmaceutical composition of 27, wherein the seal coat of the immediate release bead is at about 0.5% to about 5.0% weight/weight of the immediate release bead.

29. The pharmaceutical composition of 26, wherein the inert core of the immediate release bead comprises a microcrystalline cellulose sphere at 70% to about 95% weight/weight of the immediate release bead.

30. The pharmaceutical composition of 26, wherein the drug layer of the immediate release bead further comprises a binder and an anti-tacking agent.

31. The pharmaceutical composition of 30, wherein the drug layer of the immediate release bead comprises oxybutynin, or pharmaceutically acceptable salt thereof, at about 2% to about 15% weight/weight of the immediate release bead, the binder at about 2% to about 15% weight/weight of the immediate release bead, and the anti-tacking agent at about 1% to about 10% weight/weight of the immediate release bead.

32. The pharmaceutical composition of 30, wherein the oxybutynin, or pharmaceutically acceptable salt thereof, to binder ratio is about 1:1.

33. The pharmaceutical composition of 30, wherein the drug layer of the immediate release bead comprises oxybutynin chloride, hydroxypropyl methylcellulose (HPMC) as the binder, and talc as the anti-tacking agent.

34. The pharmaceutical composition of 33, wherein oxybutynin chloride is at about 9.8% weight/weight of the immediate release bead, hydroxypropyl methylcellulose (HPMC) is at about 9.8% weight/weight of the immediate release bead, and talc is at about 4.9% weight/weight of the immediate release bead.

35. The pharmaceutical composition of 26, wherein the plurality of immediate release beads of oxybutynin, or a pharmaceutically acceptable salt thereof, are formulated to release about 90% or more, about 95% or more, about 99% or more, or 100% of oxybutynin, or a pharmaceutically acceptable salt thereof, within about 10 minutes or less of administration of the pharmaceutical composition to a subject.

36. A method of treating hyperhidrosis comprising administering to a subject in need thereof a fixed dose pharmaceutical composition of 1.

37. The method of 36, wherein the hyperhidrosis is selected from the group consisting of primary (focal) hyperhidrosis, secondary hyperhidrosis, axillary hyperhidrosis, palmar hyperhidrosis, plantar hyperhidrosis, craniofacial hyperhidrosis, generalized hyperhidrosis, and compensatory sweating post-surgery.

38. The method of 37, wherein excessive sweating is reduced by about 5% or more, by 10% or more, by 20% or more, by 25% or more, by 50% or more, by 75% or more, by 90% or more, or by 99% or more.

39. The method of 36, wherein one or more side effects caused by oxybutynin is reduced.

40. The method of 39, wherein the one or more side effect includes dry mouth.

### EXAMPLES

### Example 1: Pilocarpine Release Profiles

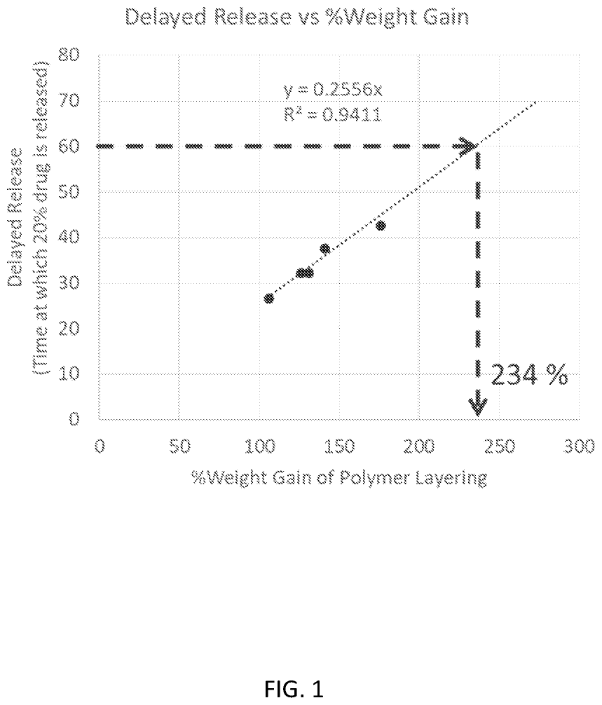

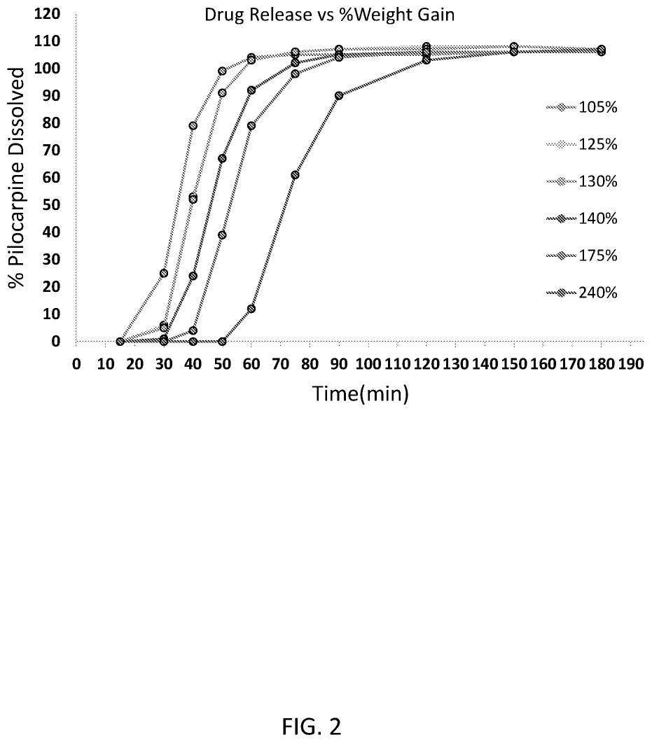

While the delayed release (t_{lag}) increases with increase in % weight gain of delayed release coating, it could slow down the release of pilocarpine after the t_{lag}. To counter this effect of slow drug release at high % weight gains, Drug:Binder ratio in the drug layer increased from 1:1 to 2:1. It was hypothesized that pilocarpine, being a high water soluble drug, can act as an osmotic agent resulting in rapid water uptake in the bead resulting in faster release. FIG. 1 shows the delayed release profile of the pilocarpine beads compared with the % weight gain of polymer layering. FIG. 2 compares the % pilocarpine dissolved over time between a range of % weight gains.

### Example 2: Oxybutynin Granulation vs. Beads

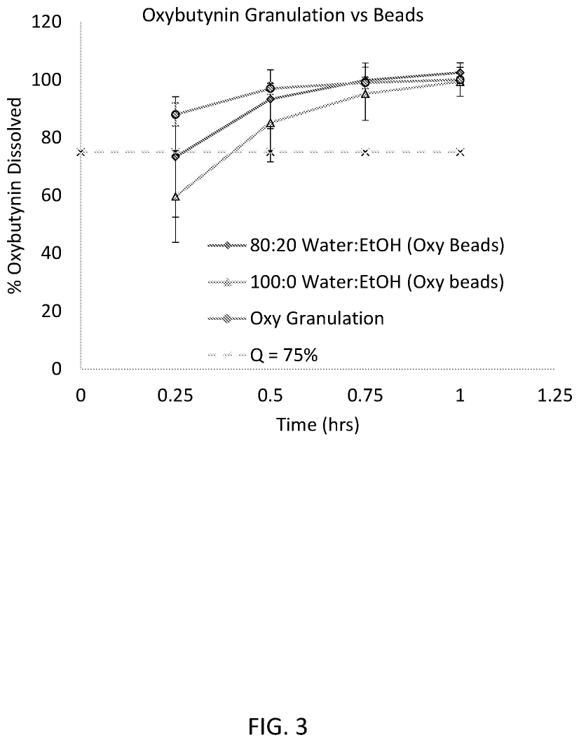

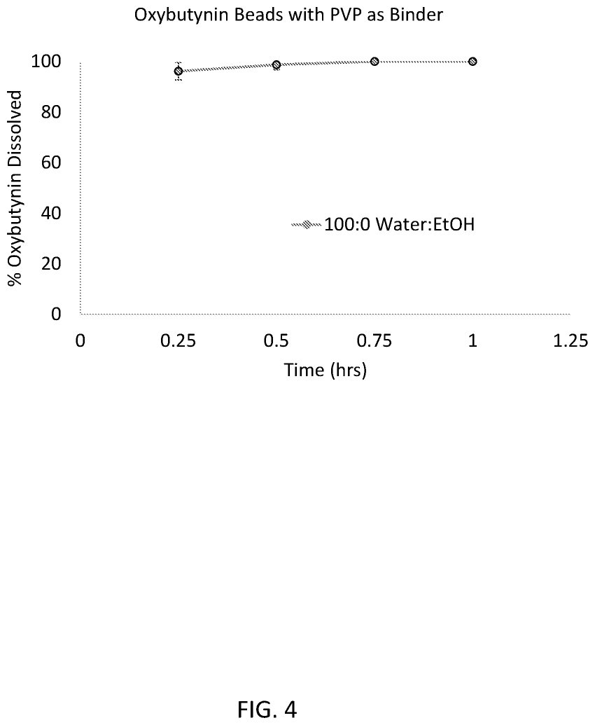

FIG. 3 compares oxybutynin beads and granulation formulations in terms of % oxybutynin dissolved over time, dashed line signifies 75% dissolved. FIG. 4 provides the dissolution profile of oxybutynin beads where the binder was changed from HPMC to PVP and changed the drug:binder ratio from 1:1 to 5:1 (less binder). Oxybutynin beads dissolution data suggests slower drug release and higher variability compared to the granulation.

Development of oxybutynin beads started with the formulation provided in Table 1.

TABLE 1
7.5 mg Oxybutynin Dose

| Component | mg/Dose | % w/w | Function |
|---|---|---|---|
| Spheres, 700 μm | 15.7 | 45.5 | Substrate |
| Oxybutynin | 7.5 | 21.8 | API |
| Chloride | | | |
| Hypromellose | 7.5 | 21.8 | Binder |
| Talc | 3.7 | 10.9 | Ant-tack Agent |
| Purified Water, | 16.5 | — | Solvent |
| USP¹ | | | |
| Alcohol¹ | 66.0 | — | Solvent |
| Total Fill per Dose | 34.4 | 100.0 | |

¹Removed during processing.

### Example 3: Multi-Particulate Technology: Spray Layered Amorphous Dispersions Using Fluid-Bed Coating for Immediate and Delayed Release

Purpose: Multiparticulates (pellets, beads, mini-tablets) offer several advantages over monolithic dosage forms including no dose dumping concerns, flexibility with strength adjustment, ease of tailoring dissolution profiles and product differentiation (life-cycle management, combination products, specialized patient populations). Amorphous solid dispersion formulations have been widely used to enhance the solubility and oral bioavailability of poorly water-soluble drugs and are generally manufactured using either spray-drying or hot-melt extrusion. However, the formulation/process development and scaling-up present a number of challenges for these techniques. In this study, amorphous solid dispersion formulations of drug, oxybutynin, were prepared by a one-step fluid-bed coating technique for immediate release (IR). Furthermore, the release of the drug from the IR beads was modified using functional coatings to achieve delayed drug release to meet in-vivo pharmacokinetic requirements for clinical studies.

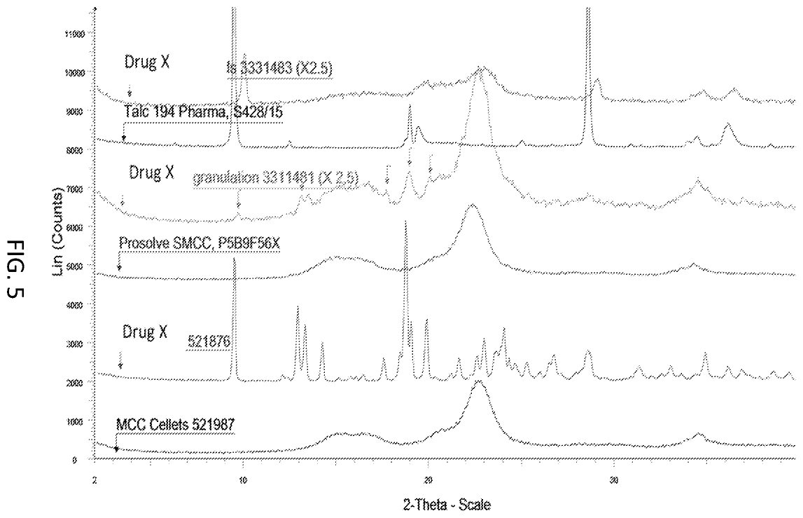

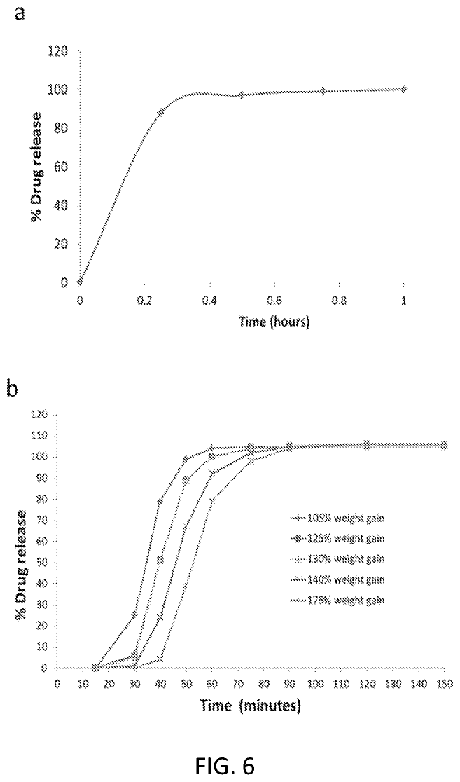

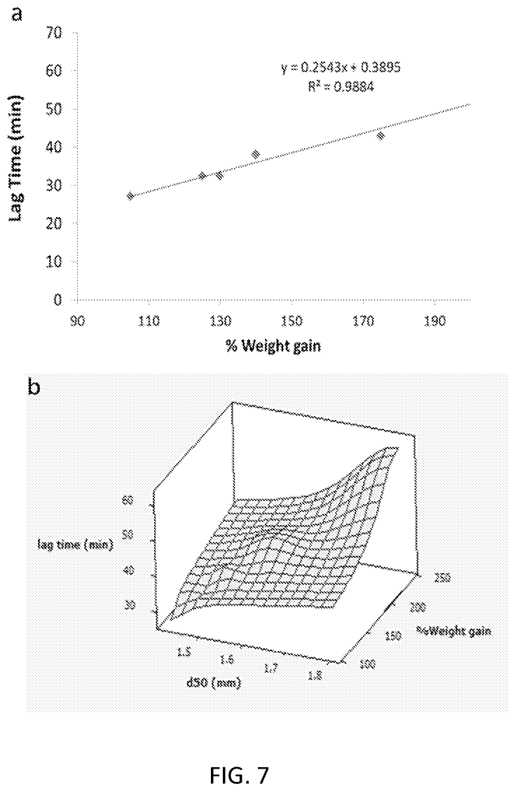

Results: The drug layered (IR) beads were prepared using two different binders, HPMC and PVP, and analyzed using XRPD for solid-state of oxybutynin (Drug X) and the results demonstrated that the drug was in the amorphous state (FIG. 5). Effect of solvent, drug:binder ratio, binder type, level of coating and level of anti-tacking agent on dissolution was studied. The dissolution of oxybutynin/HPMC solid dispersions (IR beads) provided rapid release, however the drug release was slower than expected due to the presence of talc as an anti-tacking agent (FIG. 6a). While the use of PVP as a binder resulted in faster dissolution compared to HPMC, processing with PVP was challenging and was not considered. The drug release onset time was modified using ethylcelluose barrier coating and the dissolution profiles with different barrier coating levels were evaluated (FIG. 6b). The lag time (in minutes) demonstrated a linear dependence on the % weight gain levels (50%-240%) applied to the IR beads. An empirical model was developed to correlate % (barrier-coating) weight gain with the lag time (minutes) and was used to predict the % weight gain necessary to achieve the desired lag time (FIG. 7a). Furthermore, the particle size data (d50) from Camsizer was used to estimate the growth of the beads after drug layering and functional coating. A relationship between % (barrier-coating) weight gain, d50 (mm) and lag time (minutes) was established for end-point determination (FIG. 7b).

Conclusions: In the present study, the feasibility of preparing solid dispersions using the fluid-bed coating technique was investigated, and the effects of functional coating on the dissolution was studied using empirical modeling. The results indicate that the fluid-bed coating technique has the potential use in the preparation of amorphous solid dispersions. Furthermore, fluid-bed coating technique was used to modify the release of solid dispersions using functional coatings. This technique may find application in the manufacturing and scaling-up of amorphous solid dispersion formulations.

### Example 4: Oxybutynin CL Drug Layered (DL) Beads Manufacturing Process and Formulation

Oxybutynin Cl Drug Layered (DL) Beads were developed using a smaller bead to decrease the dosage unit volume and to allow the encapsulation of the combination drug product DMVT-504, the combination of Pilocarpine HCl Delayed-immediate Release Beads and Oxybutynin Cl DL Beads in a size 3 capsule. Oxybutynin Cl Drug Layered (DL) Beads were manufactured using known processing conditions according to the formulations of Tables 2 and 3.

TABLE 2
Formulation for 10% w/w Oxybutynin Cl Drug Layered Beads

| | | | |
|---|---|---|---|
| MCS | | | Batch |
| Item | | % | Amount |
| Number | Component | Comp | Required (g) |

Substrate

| | | | |
|---|---|---|---|
| D3018 | Microcrystalline Cellulose Spheres | 75.0 | 4500 |
| | (Vivapur 500) | | |

Drug Layer Coating Suspension¹

| | | | |
|---|---|---|---|
| 72087 | Oxybutynin Cl | 10.0 | 600.0 |
| 72084 | Hydroxypropyl | 10.0 | 600.0 |
| | Methylcellulose (HPMC 606) | | |
| 10552 | Talc, USP | 5.0 | 300.0 |
| 50020 | Purified water ² | N/A | 10800 |
| 50520 | Ethyl Alcohol, 200 Proof | N/A | 2700 |
| | Dehydrated Alcohol ² | | |

| | | |
|---|---|---|
| Tablet Batch Weight | 100.0 | 6000 |

¹The drug layer coating suspension is manufactured at 10% w/w total solids. This suspension was divided into two separate portions due to lengthy processing time of approximately 12-13 hours for the coating process.
² Removed during processing.

TABLE 3
Formulation for 9.80% w/w Oxybutynin
Cl DL Beads, Seal-Coated Clear

| | | | |
|---|---|---|---|
| MCS | | | Batch |
| Item | | % | Amount |
| Number | Component | Comp | Required (g) |

Substrate

| | | | |
|---|---|---|---|
| N/A | 10% w/w Oxybutynin Cl DL Beads | 98.0 | 6000 |

Seal Coating Suspension (2% Weight Gain)¹

| | | | |
|---|---|---|---|
| 13078 | Opadry 03K19229 Clear | 1.8 | 108.0 |
| 10552 | Talc, Pharma M | 0.2 | 12.00 |
| 50520 | Purified Water, USP ² | N/A | 1880 |

| | | |
|---|---|---|
| Total Batch Weight | 100.0 | 6120 |

¹The seal coating suspension is manufactured at 6% w/w total solids. (This suspension is manufactured with a 50% excess to aid processing and is not shown in the table above.)
² Removed during processing

### DISCUSSION

Drug Layering: The goal of the Oxybutynin Cl bead optimization for Phase II clinical trial use was twofold. First, to produce a bead that could not only be machine filled but also fit as varying fill weights with varying Pilocarpine HCl bead fill weights into a size 3 hard gelatin capsule shell. Second, to reduce the size of the Oxybutynin Cl bead in relation to the Phase I bead without changing its in vivo PK profile. In order to accomplish these goals, two strategies were investigated.

The first strategy was to use less talc and binder to load the Oxybutynin Cl onto the 700 μm core bead, thereby reducing the size of the resulting beads. This strategy proved quite difficult due to an interaction between the Oxybutynin Cl and the HMPC binder. As the amount of drug content on the surface of the bead increased by reducing binder level, drug:binder ratio, the more likely the beads would stick together during processing and subsequent bulk packing. Process parameters adjustments did not eliminate this occurrence. The drug was sticky after it dried on the surface of the bead and diluting it out with binder was the only way to stop this from happening. Thus, it was not possible to directly reduce the size of the Phase I Oxybutynin Cl beads with this strategy.

This led to the second size reduction strategy and that was to use a smaller starting substrate bead. The starting substrate bead was reduced from the MCC-700 (700-1000 μm) to MCC-500 (500-750 μm). The learnings noted above were used to start the development of the MCC-500 beads in that the drug binder ratio was always set at 1:1 drug binder or higher binder levels. New issues were observed immediately mainly due to the high viscosity of the coating suspension. The Oxybutynin Cl beads would agglomerate or significant spray drying would occur during processing. In either case the resulting beads always had unacceptably low assay values. This led to the conclusion that the current process was spray drying and a portion of the active was not being coated onto the substrate.

Talc levels were increased and decreased with no effect. The percent solids content of the coating suspension was studied from 15-20% solids without effect. The composition of the solvent in the coating solutions was varied between 100% water and 83:17 water:ethyl alcohol without effect.

It was shown that it is desirable that the amount of binder be about equal to the amount of drug in the coating solution to prevent the beads from sticking together. The high binder level made the coating solution very viscous. The viscosity of the coating solution led to aggressive processing parameters such as high atomization air pressures, 2-5 Bar, to shear the liquid into fine droplets and high product temperatures, 52-58° C., to dry the sticky droplets on the beads. High atomization air pressure and product temperatures can lead to spray drying which in turn leads to low assay values. To prevent spray drying of the coating solution, the next steps were to significantly reduce the solution viscosity so less aggressive coating conditions could be used.

Stability: The following batches of Oxybutynin Cl beads were manufactured with the optimized parameters and formulation identified herein. Further, each batch was overcoated with an Opadry Clear:Talc suspension to protect the operators during encapsulation activities and to differentiate them from the Pilocarpine HCl beads.

Conclusion: Formulation of the Oxybutynin Cl Bead was optimized to produce a smaller bead product that was acceptable for automated filling processes with acceptable weight control, and that would allow sufficient volume remaining in a size 3 capsule to fill varying levels of the 8.43% w/w Pilocarpine HCl Delayed-immediate Release bead at 4 mg and 8 mg Pilocarpine HCl per capsule in the DMVT-504 product. MCS optimized the Oxybutynin Cl Drug Layered Bead first by reducing the starting substrate bead to an MCC-500 (500-750 μm) starting size to reduce the overall fill volume of the product. Secondly, a clear seal coat was added to the drug layered bead to differentiate it from the blue coated Pilocarpine HCl Delayed-immediate Release Bead contained in the DMVT-504 product and to provide a seal coat to limit the Oxybutynin Cl dust exposure for the operators during the encapsulation process.

Drug Layering: The starting substrate size was reduced from an MCC-700 bead, to an MCC-500 bead to effectively decrease the size of the final Oxybutynin Cl Drug Layered bead without effecting the release profile of the product. The drug layering formulation was not modified from the original 1:1 API to HPMC ratio with 25% w/w talc due to the sticky nature of the Oxybutynin Cl. However, to aid in processing and reduce spray drying during the coating process, the drug layering suspension was diluted from 23.4% w/w total solids with a solvent ratio of 76:24 water to ethyl alcohol to a 10% w/w total solids suspension with a solvent ratio of 80:20, water:ethyl alcohol. This change however, increased the manufacturing time by more than 2×.

The optimization of this product was successfully completed and achieved a final Oxybutynin Cl Drug Layered product that could be successfully filled into a size 3 capsule simultaneously with Pilocarpine HCl Delayed-immediate Release Beads at a 4 mg and an 8 mg dose level.

### Example 5: Pilocarpine Bead Optimization

The previously formulated delayed release coated beads were filled into a capsule along with Oxybutynin Chloride (Cl) Beads, however the Pilocarpine HCl delayed-immediate release beads were too large for acceptable automated filling process control. The following is the reformulated Pilocarpine HCl delayed-immediate release beads using smaller starting MCC spheres that would be acceptable for encapsulation on an automated capsule filler.

The Pilocarpine HCl Delayed-immediate Release (Previously formulated: Formulation A) was drug layered with 12.5% w/w Pilocarpine HCl and with a 1:1 ratio of API to HPMC onto a microcrystalline cellulose (MCC) sphere that was 700-1000 μm starting size and then polymer coated with a 1:1 ratio of ethylcellulose to hydroxypropylcellulose (Klucel EXF) with 10% dibutyl sebacate. Based on clinical data from the Phase 1 clinical trials, a targeted delayed release of 30 minutes was selected for the optimized Pilocarpine HCl formulation. Additionally, a blue HPMC coating was applied to the product. The purpose for this seal coating was two-fold: First, the seal coating was applied to possibly enhance stability of the fixed dose combination product by providing a barrier between the Pilocarpine HCl coated beads and the Oxybutynin Cl coated beads. Secondly, the blue seal coat layer would differentiate the Pilocarpine HCl delayed-immediate release beads from the Oxybutynin Cl Drug Layered Beads.

### Pilocarpine HCl Drug Layering

To achieve a smaller bead formulation, three different sized starting MCC spheres were evaluated. MCC Spheres with various sizes of 200-350 μm (MCC-200), 350-500 μm (MCC-350), and 500-700 μm (MCC-500) were each coated with 10% w/w Pilocarpine HCl and with a 5:1 ratio of API to HPMC. The MCC-500 sphere was selected as the most viable substrate based on ease of manufacturing and acceptable final coated bead size.

Pilocarpine HCl Drug Layered (DL) Beads were manufactured using known processing conditions according to the formulations of Table 4 (10%), Table 5(8.77%), and Table 6 (8.43%).

TABLE 4
Formulation for 10% w/w Pilocarpine HCl Drug Layered Beads

| | | | |
|---|---|---|---|
| MCS | | | Batch |
| Item | | % | Amount |
| Number | Component | Comp | Required (g) |

Substrate

| | | | |
|---|---|---|---|
| 72083 | Microcrystalline Cellulose | 85.0 | 6800 |
| | Spheres | | |
| | (Cellets 500) | | |

Drug Layer Coating Suspension¹

| | | | |
|---|---|---|---|
| 72086 | Pilocarpine HCl | 10.0 | 800.0 |
| 72084 | Hydroxypropyl Methylcellulose | 2.0 | 160.0 |
| | (HPMC 606) | | |
| 10552 | Talc, USP | 3.0 | 240.0 |
| 50020 | Purified water ² | N/A | 5600 |

| | | |
|---|---|---|
| Tablet Batch Weight | 100.0 | 8000 |

¹The drug layer coating suspension is manufactured at 17.65% w/w total solids.
² Removed during processing

TABLE 5
Formulation for 8.77% w/w Pilocarpine
HCl Delayed-immediate Release Beads

| | | | |
|---|---|---|---|
| MCS | | | Batch |
| Item | | % | Amount |
| Number | Component | Comp | Required (g) |

Substrate

| | | | |
|---|---|---|---|
| N/A | 10% w/w Pilocarpine HCl DLB | 87.72 | 8000 |

Polymer Layer Coating Solution (14% Weight Gain) ¹

| | | | |
|---|---|---|---|
| 10631 | Hydroxypropylcellulose | 2.76 | 252.0 |
| | USP/NF (Klucel EXF) | | |
| 11991 | Ethylcellulose, USP/NF/EP | 8.29 | 756.0 |
| 72085 | Dibutyl Sebacate, USP/NF | 1.23 | 112.0 |
| 50520 | Ethyl Alcohol, 200 Proof | N/A | 10080 |
| | Dehydrated Alcohol, | | |
| | USP ² | | |

| | | |
|---|---|---|
| Total Batch Weight | 100.0 | 9,120 |

¹ The polymer layer coating solution is manufactured at 10% w/w total solids.
² Removed during processing

TABLE 6
Formulation for 8.43% w/w Pilocarpine HCl
Delayed-immediate Release Seal Coated Beads

| | | | |
|---|---|---|---|
| MCS | | | Batch |
| Item | | % | Amount |
| Number | Component | Comp | Required (g) |

Substrate

| | | | |
|---|---|---|---|
| N/A | 8.77% w/w Pilocarpine HCl | 96.15 | 9120 |
| | Delayed-immediate Release Beads | | |

Seal Layer Coating Suspension (4% Weight Gain)¹

| | | | |
|---|---|---|---|
| 13077 | Opadry 03K105035 Blue | 3.85 | 364.8 |
| 50520 | Purified Water, USP | N/A | 3283.2 |

| | | |
|---|---|---|
| Total Batch Weight | 100.0 | 9484.8 |

¹The Seal layer coating suspension is manufactured at 10% w/w total solids. (This suspension is manufactured with a 50% excess to aid processing and is not shown in the table above.)
² Removed during processing

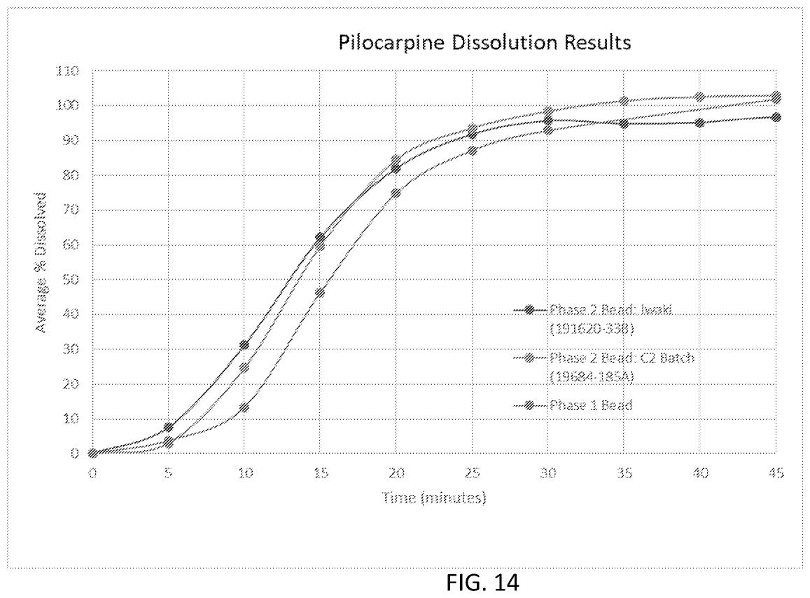

The formulation and process parameters used to manufacture batches of Pilocarpine HCl Delayed-immediate Release Beads with API from C2 were completely applicable to those manufactured with Iwaki API. Important qualities shared by both sources of API include rapid solubility in water to prepare the drug layering solution and equivalent dissolution profile of the finished beads as shown below in FIG. 14.

### DISCUSSION

To reduce the dosing volume of the Pilocarpine HCL Delayed-immediate Release Bead and allow for acceptable weight control on automated encapsulating equipment, the pilocarpine beads were reformulated using two separate approaches. The first approach was to reduce the size of the pilocarpine HCL drug layered beads. The second approach was to reduce the coating level of the functional polymer coat. Additionally, a seal coat was applied to both separate the functional coat of the Pilocarpine HCl Delayed-immediate Release Bead from the Oxybutynin Chloride Bead and to provide visual differentiation between the two beads.

All of these modifications were incorporated in such a way that the resulting bead matched or was slightly faster than the dissolution profile obtained from the Phase I pilocarpine HCl beads. Thus, requirements of not more than 30% released in 10 minutes and not less than 85% released at 45 minutes must be met.

Pilocarpine HCl Drug Layered Beads Process Development: The following ideas were considered to reduce the size of the pilocarpine drug layered beads.
Reduce the starting microcrystalline cellulose (MCC) bead size.
Reduce drug load on the MCC beads from 20% w/w to 10% w/w.
Reduce to binder level in the bead formulation from 1:1 to 5:1 drug:binder.

Three different sizes of MCC spheres were evaluated during the reformulation of the product while simultaneously reducing the binder level to 5:1 drug:binder and reducing the drug load on the microcrystalline cellulose beads to 10% w/w. MCC-200 (200-350 μm), MCC-350 (350-500 μm), and MCC-500 (500-750 μm) were all coated with a 10% w/w drug layer with a drug to HPMC ratio of 5:1.

Pilocarpine HCl Polymer Layer Process Development: After decreasing the size of the pilocarpine HCl drug layered beads as noted above the next phase of development focused on reducing the coat level of the functional polymer coat. The following ideas were incorporated into the functional polymer coat to reduce the size of the Pilocarpine HCl Delayed-immediate Release beads.
Vary the size in the drug layered beads, 350 or 500 μm.
Vary the coat composition, ethylcellulose:hydroxypropyl cellulose ratio.
Vary the weight gain of the functional coat.

First, it is important to consider what effect drug layered bead size will have on polymer coat requirements. A brief discussion of the effects of bead size on dissolution profile is presented below.

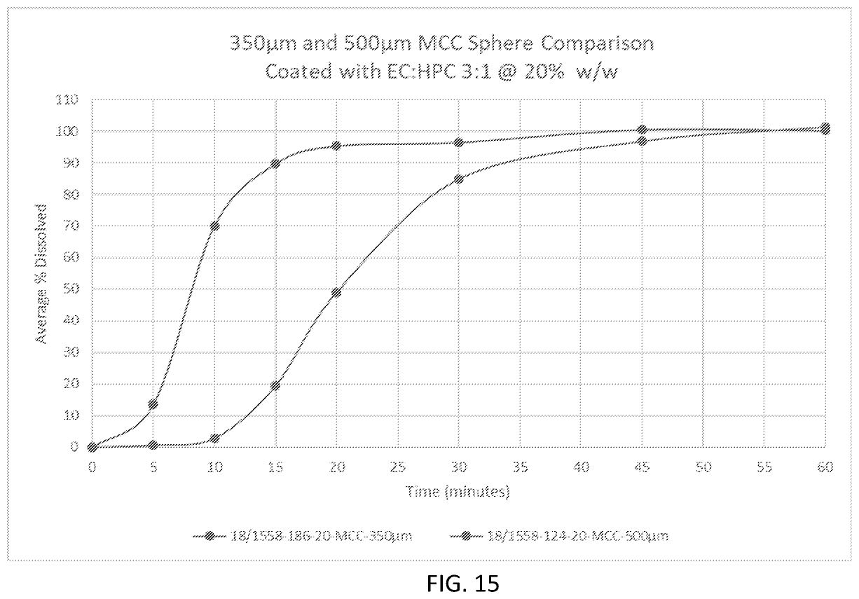

Batches 18/1557-15C and 18/1556-65A drug layered beads were additionally coated with a polymer layer that contained a 3:1 ratio of EC to HPC and 10% dibutyl sebacate. FIG. 15 below shows the effect on dissolution respective only to the size of the starting substrate. Other factors were considered in the decision to select the 500 μm drug layered beads and they are shown in Table 7.

TABLE 7
Pilocarpine HCl Delayed-immediate Release Bead Size Acceptance Criteria

| Sphere | | | |
|---|---|---|---|
| Size | Parameters/Attributes | Advantages | Disadvantages |
| 500 μm | Faster spray rate results in shorter process time | X | |
| | to make beads | | |
| | Lighter polymer coats result in shorter process | X | |
| | time to make beads | | |
| | Optimized bead resulted in greater than 10x | X | |
| | increase of number of beads per dose for better | | |
| | CU | | |
| 350 μm | Slower spray resulting in longer process time | | X |
| | to make beads | | |
| | Heavier polymer coats result in a longer | | X |
| | process time to make beads | | |
| | Optimized bead resulted in greater than10x | X | |
| | increase of number of beads per dose for better | | |
| | CU | | |

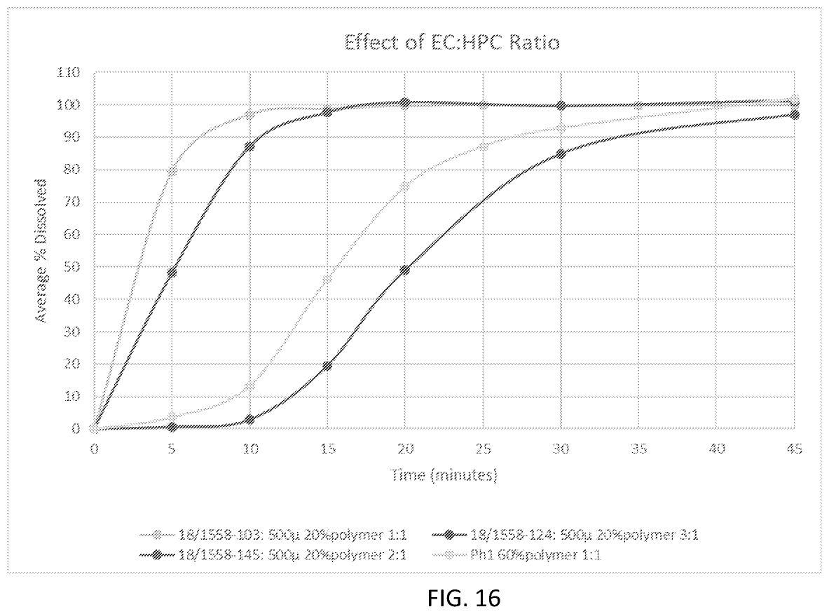

The second factor explored to reduce the size of the Pilocarpine HCl DR Beads was to vary the polymer coat composition. In these experiments, the ratio of ethylcellulose, water insoluble polymer, to hydroxypropyl cellulose (HPC), water soluble polymer, was varied from 1:1, 2:1 and 3:1. As shown in FIG. 16, as ethylcellulose level increases from 1:1 to 3:1, dissolution rate decreases. Note that this relationship is not linear as a polymer composition of 3:1 is much slower than either 1:1 or 2:1. Thus, it would be possible to use considerably less polymer coat and match the Phase I pilocarpine HCl bead profile. As the Phase I bead was manufactured with a 60% w/w coat of 1:1 ethylcellulose:HPC, a similar pilocarpine bead with a coat composition of 3:1 and a coat level of less than 20% w/w would be significantly smaller. For this reason, a 3:1 ratio of ethylcellulose:HPC was incorporated into the final pilocarpine HCl DR bead design.

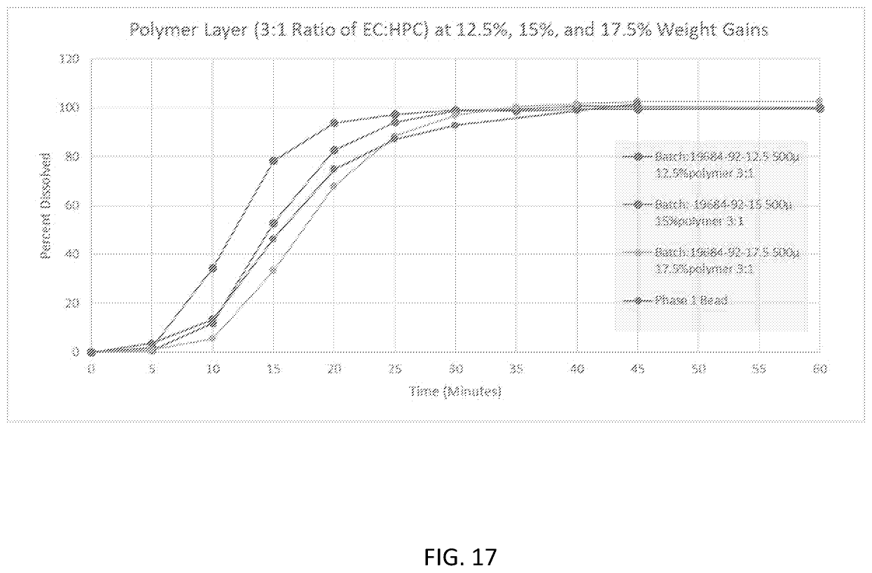

With the factors of starting bead size set to 500 μm and the coat composition set to 3:1 ethylcellulose:HPC, the third factor to set was the coat level. FIG. 17 depicts the relationship of dissolution rate as a function of coat level. As coat level increases, the dissolution rate decreases. A coat level of 12.5 to 17.5% w/w bracketed the Phase I dissolution profile.

The final determination of the polymer coating level for the product was based primarily on the analytical data collected from Batch 19684-92. This batch was coated with a polymer layer with a 3:1 ratio of EC:HPC to a coating level of 17.5% and additionally samples were removed and tested at 12.5%, and 15% weight gains. The 15% and 17.5% coating levels both have similar release profile curves to the release rate of the Phase 1 CTM targeted release profile. The 15% coating level released slightly faster. (Refer to FIG. 16) Based on the theory that the seal coating layer should slightly delay the release of the drug, a polymer coating level of 14% was selected.

Pilocarpine HCl Delayed-immediate Release Seal Coating Layer Process Development: To provide a barrier between the Pilocarpine HCl beads and the Oxybutynin Cl beads in the DMVT-504 product and to distinguish the Pilocarpine HCl Delayed-immediate Release Bead from the Oxybutynin Cl Bead, a seal coating of Opadry 03K105035 Blue was coated onto the Pilocarpine HCl Delayed-immediate Release Bead. Batch 19684-22 was coated using a 10% w/w total solids suspension of Opadry 03K105035 Blue and purified water. The batch was coated to a 4% weight gain and additional samples were taken from the batch at 2% and 3% coating weight gains.

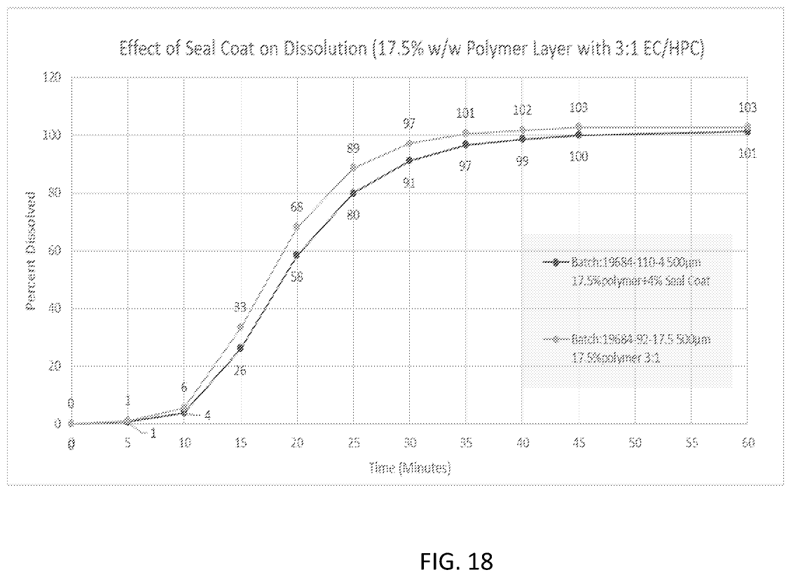

Pilocarpine HCl Delayed-immediate Release Beads, Batch 19684-92-17.5 was coated with a 4% weight gain of Opadry. This batch was tested for dissolution to determine the effect of the seal coat layer on the product dissolution. As expected, the seal coated batch released slightly slower than the uncoated batch. Refer to FIG. 18.

Conclusion: The goal for Pilocarpine Bead Optimization was to produce a product that could be encapsulated on an automated encapsulation machine with acceptable weight control. Prior attempts were formulated with 12.5% w/w Pilocarpine HCl delayed-immediate release beads that were too large for acceptable automated filling process control. Therefore, the Pilocarpine HCl delayed-immediate release beads were reformulated using smaller starting MCC spheres that would be acceptable for encapsulation on an automated capsule filler into a size 3 hard gelatin capsule.

The Pilocarpine HCl Drug Layered Bead reformulated by decreasing the starting microcrystalline cellulose substrate from the MCC 700 (700-1000 μm) to a smaller MCC-500 (500-750 μm) substrate. The drug layering process was further modified by reducing the drug loading from 20% to 10% w/w on the drug layered beads to increase the number of beads needed per capsule. Additionally, the HPMC binder level was decreased from 1:1 to a 5:1 drug:binder ratio. This resulted in a decrease in the processing time and further reduced the overall size of the finished beads. The drug layer formulation changes were considered acceptable for the reformulation of a smaller Pilocarpine HCl Drug Layered Bead. The polymer layer was reformulated from a 60% w/w coating of a 1:1 EC:HPC polymer layer to a 14% w/w coating of a 3:1 EC:HPC polymer layer to achieve the same target a similar dissolution release that was observed for the Phase I 12.5% w/w Pilocarpine HCl Delayed-immediate Release Beads. The polymer layer formulation changes were considered acceptable for the reformulation of a smaller Pilocarpine HCl Delayed-immediate Release Bead.

To provide a barrier between the Pilocarpine HCl beads and the Oxybutynin Cl beads in the product and to distinguish the Pilocarpine HCl Delayed-immediate Release Bead from the Oxybutynin Cl Bead, a 4% weight gain seal coating of Opadry 03K105035 Blue was coated onto the Pilocarpine HCl Delayed-immediate Release Beads. A coating weight gain of 4% was determined to be acceptable based on the visual observations.

The reformulated 8.43% w/w Pilocarpine HCl Delayed-immediate Release Seal Coated Beads were successfully encapsulated on an automated encapsulation machine with acceptable weight control. These encapsulated batches were packaged and placed on stability.

### Example 6: Excipient Compatibility

The purpose of the excipient compatibility study is to rank order eight multi-component blends containing both Pilocarpine Hydrochloride (HCl) and Oxybutynin Chloride in terms of change in total impurities. Control blends of both APIs together and a placebo blend were also studied. The excipient compatibility study was set up in the manner of a stability study at 40° C./75% RH and 50° C./AMB (Ambient) conditions. The blends were stored at very rigorous conditions to elicit significant changes in impurity levels for a period of 8 weeks. Sufficient change in the desired response was seen in this time period such that a further test point at 12 weeks was not needed. Additional testing included the water content of each multi-component blend and an optional assay value at each of the timepoints. Table 8 provides the formulations tested.

TABLE 8
Formulations

| | A | B | C | D | E | F | G | H | Control | |
|---|---|---|---|---|---|---|---|---|---|---|
| | Weight | Weight | Weight | Weight | Weight | Weight | Weight | Weight | Weight | Placebo |
| | (mg)/% | (mg)/% | (mg)/% | (mg)/% | (mg)/% | (mg)/% | (mg)/% | (mg)/% | (mg)/% | Target |
| Component | w/w | w/w | w/w | w/w | w/w | w/w | w/w | w/w | w/w | wt (g) |
| Oxybutynin | 7.5/5.0 | 7.5/5.0 | 7.5/5.0 | 15.0/100 | 7.5/5.0 | 7.5/5.0 | 7.5/5.0 | 7.5/5.0 | 75.0/50.0 | |
| Cl | | | | | | | | | | |
| Pilocarpine | 7.5/5.0 | 7.5/5.0 | 7.5/5.0 | 15.0/100 | 7.5/5.0 | 7.5/5.0 | 7.5/5.0 | 7.5/5.0 | 75.0/50.0 | |
| HCl | | | | | | | | | | |
| MCCPH102 | 119.3/79.5 | | 89.3/59.5 | 63.0/42.0 | | | 48.0/320 | 95.3/63.5 | | 3 |
| HPMCPC606 | 6.0/4.0 | | | 6.0/4.0 | | | 6.0/4.0 | | | 3 |
| Sodium | 3.0/2.0 | | 3.0/2.0 | | 3.0/2.0 | 6.0/4.0 | | 30.0/2.0 | | 3 |
| startch | | | | | | | | | | |
| glycolate | | | | | | | | | | |
| Magnesium | 0.8/0.5 | | 0.8/0.5 | | 0.8/0.5 | | | 0.8/0.5 | | 3 |
| stearate | | | | | | | | | | |
| Opadry | 6.0/4.0 | | | 6.0/4.0 | | 6.0/4.0 | 6.0/4.0 | | | 3 |
| light blue | | | | | | | | | | |
| Dicalcium | | 116.3/77.5 | | | 95.3/63.5 | | | | | 3 |
| phosphate | | | | | | | | | | |
| Silicon | | 0.8/0.5 | | | | | | | | 3 |
| dioxide | | | | | | | | | | |
| Providone | | 6.0/4.0 | | | | | | | | 3 |
| K29-32 | | | | | | | | | | |
| Crospovidone | | 3.0/2.0 | | | | | | | | 3 |
| XL 10 | | | | | | | | | | |
| Stearic acid | | 3.0/2.0 | | | | 3.0/2.0 | | | | 3 |
| Opadry clear | | 6.0/4.0 | 6.0/4.0 | | 6.0/4.0 | | | 6.0/4.0 | | 3 |
| Startch 1500 | | | 30.0/20.0 | | | | | | | 3 |
| HPC | | | 6.0/4.0 | 15.0/10.0 | | | 15.0/100 | | | 3 |
| Talc | | | | 15.0/10.0 | | | 15.0/100 | | | 3 |
| Ethycellulose | | | | 15.0/100 | | | 45.0/30.0 | | | 3 |
| Fast Flo | | | | | 30.0/200 | 120.0/80.0 | | 30.0/200 | | 3 |
| Lactose | | | | | | | | | | |

Appearance, LOD, API analysis, pilocarpine impurities, and oxybutynin impurities were evaluated.

Discussion: The Oxybutynin Chloride was found to be very stable at both storage conditions for all formulations and timepoints. The Pilocarpine HCl had good stability at the 50° C./Ambient condition. All formulations of Pilocarpine HCl degraded at the 40° C./75% RH condition and is therefore believed to be a water mediated pathway. The most stable formulations at the 40° C./75% RH condition were formulations D and G. The assay values were found to be sporadic at the initial condition. An investigation revealed this was due to the variable nature of the blending procedure which, in turn, affected variability of the assay.

Conclusion: The formulation degradation results show that the preferred formulations are: Formulation G and D, followed by formulations C, F, H, B, A and E.

### Example 7: DMVT-504 Oxybutynin/Pilocarpine Capsules: Encapsulation Optimization

The following provides information regarding the development of a process using a smaller bead to decrease the dosage unit volume to allow for the encapsulation of the combination drug product DMVT-504 Pilocarpine HCl Delayed-immediate Release Bead and Oxybutynin Cl DL Bead in a size 3 capsule.

The DMVT-504 Capsules for Phase 1 of this project contained both drug layered Oxybutynin Cl beads and Pilocarpine HCl Delayed-immediate Release beads coated onto microcrystalline cellulose (MCC-700) sphere that was 700-1000 μm starting size. This drug layered product was not suitable for automated filling machines due to the drug loading, too few beads per dose, and size of each bead. MCS was contracted to modify and develop a formulation that would be suitable to manufacture the DMVT-504 combination product containing both, Pilocarpine HCl and Oxybutynin Cl filled with an automated encapsulating machine into a size 3 capsule at various dosage strengths.

9.80% w/w Oxybutynin Cl Drug Layered Beads: To achieve a smaller bead formulation, the Oxybutynin Cl formulation was modified to a 9.80% w/w Oxybutynin Cl and with a 1:1 ratio of API to HPMC coated onto a microcrystalline cellulose (MCC-500) sphere that was 500-750 μm starting size. The drug layered Oxybutynin bead was then coated with a 2% weight gain of Opadry 03K19229 Clear with additional talc added at a 90:10 ratio. The seal coating was added to further differentiate the Oxybutynin Bead from the Pilocarpine Bead which was blue in color. Additionally, the seal coat offered the added protection to the operators by reducing the generation of dust during the encapsulation process.

8.43% w/w Pilocarpine HCl Delayed-immediate Release Beads: To achieve a smaller bead formulation, the Pilocarpine HCl formulation was modified to an 8.43% w/w Pilocarpine HCl and with a 5:1 ratio of API to HPMC onto a microcrystalline cellulose (MCC-500) sphere that was 500-750 μm starting size. The drug layered bead was then coated with a 3:1 ethylcellulose:hydroxypropyl cellulose ratio polymer layer coated to a 14% weight gain to achieve a release profile similar to the target Phase I Formulation A. The bead was then coated with 4% weight gain of Opadry 03K105035 Blue cosmetic coating to reduce the generation of dust during the encapsulation process and to distinguish the Pilocarpine HCl bead from the Oxybutynin Cl bead.

DMVT-504 Capsules, Oxybutynin Cl, 6 mg, and Pilocarpine HCl Delayed-immediate Release, 4 mg and 8 mg: The DMVT-504 Capsules were manufactured on the IMA Zanasi 16E Plus encapsulating machine. This machine has an operating speed that is adjustable from 3 k/Hr to 16 k/Hr. Due to batch sizes, initial trials were operated at 3 k/Hr but eventually that speed was increased to 10 k/Hr to decrease the product weight variation. The capsules were size 3 blue hard gelatin and were filled with both the 9.80% w/w Oxybutynin Cl beads and the 8.43% w/w Pilocarpine HCl Delayed-immediate Release beads using Size 4 or Size 5 Dosators. The Size 5 dosator was eventually selected given the low fill volume of each of the component beads.

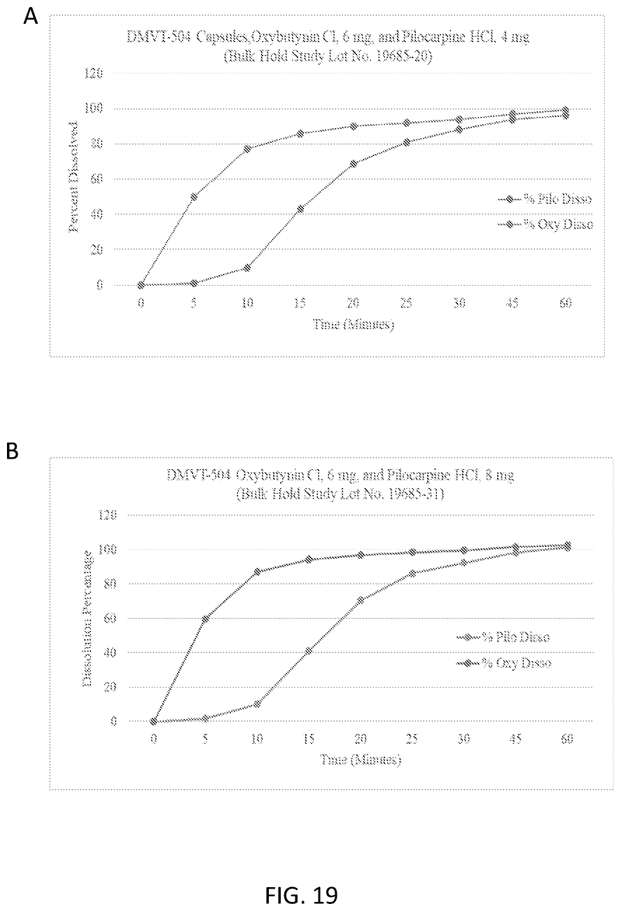

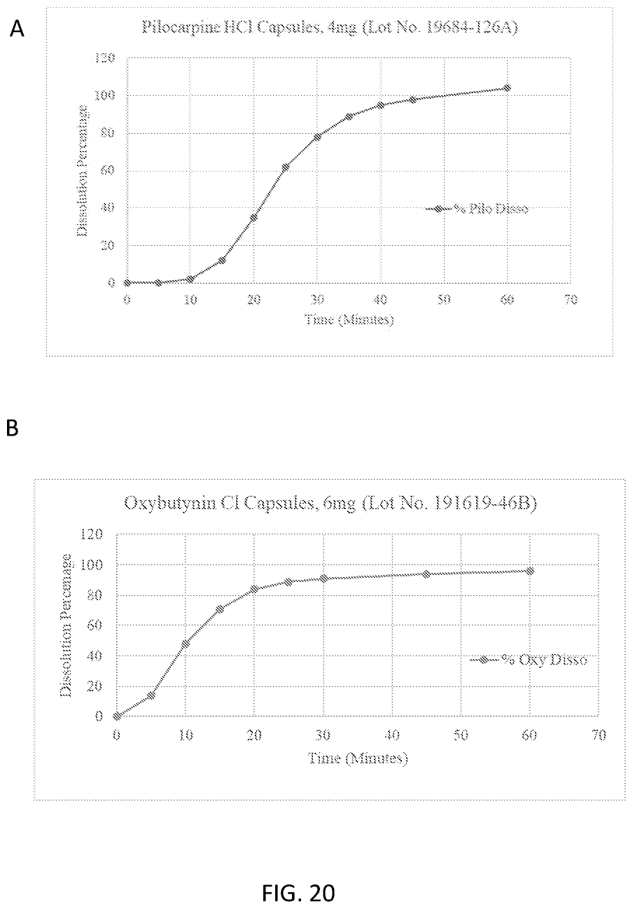

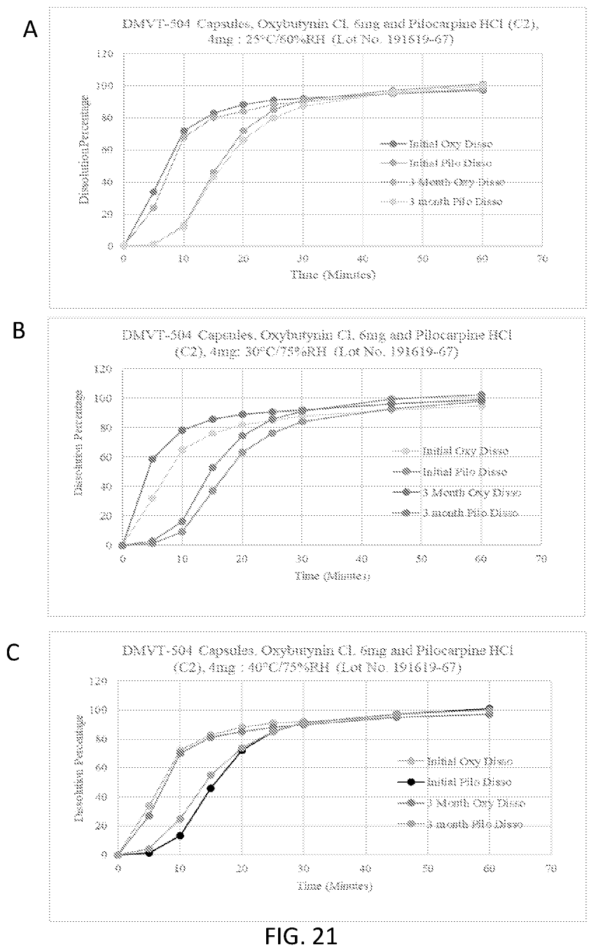

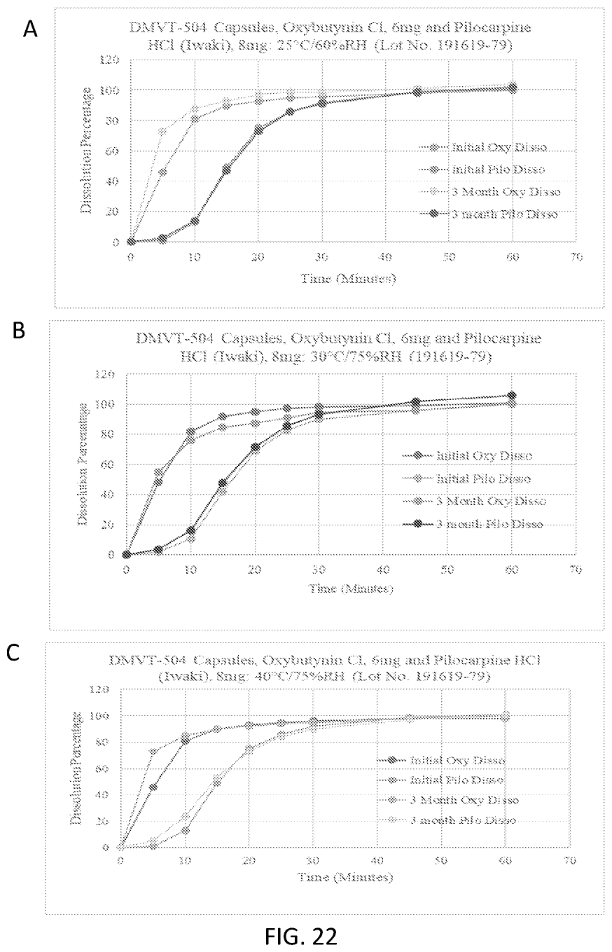

The following batches of Capsules were manufactured with the optimized parameters and formulation identified within this report. FIGS. 19-22 show the dissolution profile for each of the stability conditions and time points tested.

Discussion: The goal of this project was to fill Size 3 capsules with 6 mg of Oxybutynin Cl in an immediate release formulation and 4 mg, 6 mg, or 8 mg of Pilocarpine HCl in a delayed-immediate release formulation using an automated encapsulation machine with acceptable weight control for each active ingredient.

The batches manufactured for the DMVT-504 product were placed on developmental stability at 25° C./60% RH, 30° C./75% RH, and 40° C./75% RH storage conditions. The assay, content uniformity, and dissolution data from this report has acceptable results at all storage conditions for both Pilocarpine and Oxybutynin active ingredients.

Conclusion: The objective of the DMVT-504 Oxybutynin/Pilocarpine Capsules, Phase 2 Encapsulation optimization was to fill Size 3 capsules with 6 mg of Oxybutynin Cl and 4 mg, 6 mg, or 8 mg of Pilocarpine HCl using an automated encapsulation machine with acceptable weight control, assay, and content uniformity for each active ingredient. Given the small dose volume required for each of the optimized active dosage forms, it was determined that Size 5 dosators would be employed to encapsulate the product to ensure adequate weight control for each dosage strength.

### Example 7: A Phase I, Partially-Randomized, 7-Way Crossover Study to Evaluate the Pharmacokinetics, Safety, Tolerability and Food Effect of Single Oral Doses of Different Fixed-Dose Combination Formulations of RVT-504 in Healthy Volunteers

In a Phase 1 clinical study (study number DVMT-504-1001), plasma levels of piolocarpine, R-oxybutynin and S-oxybutynin were measured. While the R- and S-enantiomers of oxybutynin were measured, the R-oxybutynin enantiomer is responsible for approximately 9000 of the activity of the drug and the S-oxybutynin enantiomer only approximately 10% of the activity. See Table 12.

TABLE 12
Protocol

| | |
|---|---|
| Study Phase | Phase 1 |
| Investigational Product | 5 formulations of RVT-504, a fixed-dose combination |
| | (FDC) of oxybutynin and pilocarpine |
| Reference Products | Oxybutynin chloride |
| | Pilocarpine hydrochloride |
| Objectives | Primary |
| | To evaluate the pharmacokinetics (PK) of single oral |
| | doses of different FDC formulations of RVT-504, |
| | administered in the fasted state, relative to the PK of |
| | single doses of oxybutynin and pilocarpine given 30 |
| | minutes apart, administered in the fasted state. |
| | To evaluate the effect of food on the PK of a selected |
| | FDC formulation of RVT-504. |
| | To assess the safety and tolerability of different FDC |
| | formulations of RVT-504. |
| | Secondary |
| | To assess the pharmacodynamics (PD), as measured by |
| | an assessment of dry mouth, of single oral doses of |
| | different FDC formulations of RVT-504 relative to |
| | single oral doses of oxybutynin and pilocarpine given 30 |
| | minutes apart. |
| Target Population | Healthy adult subjects |
| Number of Subjects | Approximately 16 (12 evaluable) |
| Planned | |
| Number of Study | 1 center in US |
| Centers Planned | |
| Study Design | This is a partially-randomized, 7-way crossover study to |
| | assess the PK, safety, tolerability, food effect and PD of |
| | different FDC formulations of RVT-504 relative to |
| | oxybutynin and pilocarpine given 30 minutes apart. Period |
| | 1 will evaluate the PK, safety, tolerability and PD of single |
| | oral doses of oxybutynin and pilocarpine given 30 minutes |
| | apart. Periods 2 through 6 will evaluate the PK, safety, |
| | tolerability and PD of single oral doses of five different |
| | FDC formulations of RVT-504 with differing pilocarpine |
| | release characteristics. Period 7 will evaluate the effect of |
| | food on one of the five FDC formulations of RVT-504. The |
| | study will be partially-randomized as Periods 1 and 7 will |
| | be fixed sequence and Periods 2 through 6 will be |
| | randomized. |
| | Subjects will reside in the clinic for the duration of dosing. |
| | Single doses will be administered in 48-hour intervals on |
| | the mornings of Days 1, 3, 5, 7, 9, 11, and 13. PK samples |
| | will be collected pre-dose and at 0.5, 1, 1.5, 2, 2.5, 3, 4, 6, 8 |
| | and 12 hours post-dose on Days 1, 3, 5, 7, 9, 11 and 13. PK |
| | samples will be analyzed for concentrations of oxybutynin |
| | (R— and S—), desethyloxybutynin (R— and S—), pilocarpine, |
| | and pilocarpic acid. Dry mouth assessments will be made |
| | pre-dose and at 1, 2, 3, 4, 6, and 8 hours post-dose on Days |
| | 1, 3, 5, 7, 9, 11 and 13. Each subject will participate for up |
| | to approximately six weeks including screening and |
| | treatment. |
| Duration of Treatment | Single oral dose administration on seven separate occasions |
| Criteria for Evaluation | Primary |
| (Endpoints) | Pharmacokinetic parameters of oxybutynin (R— and |
| | S—), desethyloxybutynin (R— and S—), pilocarpine, |
| | and pilocarpic acid in plasma for each period as data |
| | permit, including: Cₘₐₓ, tₘₐₓ, AUCₗₐₛₜ, AUC_{inf}, t_{1/2}, |
| | CL/F, and V_{z}/F. |
| | Safety parameters including frequency and severity |
| | of adverse events (local and systemic), vital signs, |
| | and laboratory values. |
| | Secondary |
| | Incidence and severity of dry mouth |
| Statistical Methods | Safety, PK, and PD data will be presented in tabular and/or |
| | graphical format and summarized descriptively. Non- |
| | compartmental PK analysis will be performed for |
| | oxybutynin (R— and S—), desethyloxybutynin (R— and S—), |
| | pilocarpine, and pilocarpic acid in plasma for each period. |

Tables 13 and 14 provide the formulations of the pilocarpine and oxybutynin beads, respectively, evaluated in the trial and compounded into size 1 capsules.

TABLE 13
Composition of Pilocarpine Delayed-Release Beads
Core Bead

| | Formulation ID | | | | | | | | | | | |
|---|---|---|---|---|---|---|---|---|---|---|---|---|
| | 3A | | 3B | | 3C | | 3D | | 3E | | | |
| | mg/ | % | mg/ | % | mg/ | % | mg/ | % | mg/ | % | | |
| Ingredient | Capsule | W/W | Capsule | W/W | Capsule | W/W | Capsule | W/W | Capsule | W/W | Function | Specification |
| Microcrystalline | 18.75 | 31.25 | 18.75 | 26.32 | 18.75 | 20.83 | 18.75 | 17.86 | 18.75 | 16.12 | Substrate | NF |
| Cellulose Spheres | | | | | | | | | | | | |
| (700 μm) | | | | | | | | | | | | |
| Pilocarpine | 7.50 | 12.5 | 7.50 | 10.53 | 7.50 | 8.33 | 7.50 | 7.14 | 7.50 | 6.45 | API | USP |
| Hydrochloride | | | | | | | | | | | | |
| Hypromellose | 7.50 | 12.5 | 7.50 | 10.53 | 7.50 | 8.33 | 7.50 | 7.14 | 7.50 | 6.45 | Binder | USP |
| Talc | 3.75 | 6.25 | 3.75 | 5.26 | 3.75 | 4.17 | 3.75 | 3.57 | 3.75 | 3.22 | Anti-tack | USP |
| | | | | | | | | | | | Agent | |
| Purified Water^{a} | QS | — | QS | — | QS | — | QS | — | QS | — | Solvent | USP |
| Total Core | 37.5 | 62.50 | 37.5 | 52.64 | 37.5 | 41.66 | 37.5 | 35.71 | 37.5 | 32.24 | | |

Polymer Coat

| Coating Weight Gain | 60% of Core | | 90% of Core | | 140% of Core | | 180% of Core | | 210% of Core | | | |
|---|---|---|---|---|---|---|---|---|---|---|---|---|
| Hydroxypropyl | 10.25 | 17.08 | 15.37 | 21.57 | 23.9 | 26.56 | 30.75 | 29.29 | 35.88 | 30.85 | Pore | NF |
| Cellulose | | | | | | | | | | | Former | |
| Ethylcellulose | 10.25 | 17.08 | 15.37 | 21.57 | 23.9 | 26.56 | 30.75 | 29.29 | 35.88 | 30.85 | Polymer | NF |
| Dibutyl | 2.00 | 3.33 | 3.01 | 4.22 | 4.70 | 5.22 | 6.00 | 5.71 | 7.00 | 6.02 | Plasticizer | NF |
| Sebacate | | | | | | | | | | | | |
| Alcohol^{a} | QS | — | QS | — | QS | — | QS | — | QS | — | Solvent | USP |
| Total Polymer | 22.50 | 37.49 | 33.75 | 47.36 | 52.50 | 58.34 | 67.50 | 64.29 | 78.75 | 67.72 | | |
| Coat (mg) | | | | | | | | | | | | |
| Total Bead Fill | 60.00 | 100.0 | 71.25 | 100.0 | 90.00 | 100.0 | 105.0 | 100.0 | 116.3 | 100.0 | | |
| Weight (mg) | | | | | | | | | | | | |

TABLE 14
Oxybutynin chloride bead
7.5 mg Oxybutynin Chloride Dose

| Component | mg/Dose | % w/w | Function |
|---|---|---|---|
| Spheres, 700 μm | 56.25 | 75.00 | Substrate |
| Oxybutynin | 7.50 | 10.00 | API |
| Chloride | | | |
| Hypromellose | 7.50 | 10.00 | Binder |
| Talc | 3.75 | 5.00 | Ant-tack Agent |
| Purified Water, | QS | — | Solvent |
| USP¹ | | | |
| Alcohol¹ | QS | — | Solvent |
| Total Fill per Dose | 75.0 | 100.0 | |

¹Removed during processing.

Table 15 presents the R-oxybutynin enantiomer data: The Cₘₐₓ was 4.239 ng/mL when the oxybutynin plus pilocarpine tablets were administered compared to 5.349 ng/mL when the DMVT-504 Formulation 3A capsule was administered.

TABLE 15
Summary of Plasma R-oxybutynin Following Administration of 7.5 mg Oxybutynin Chloride and
7.5 mg Pilocarpine Hydrochloride Tablets and Various FDCs Under Fed and Fasted Conditions

| | | DMVT-504 | DMVT-504 | DMVT-504 | DMVT-504 | DMVT-504 | DMVT-504 |
|---|---|---|---|---|---|---|---|
| Pharmacokinetic | Oxybutynin + | Formulation | Formulation | Formulation | Formulation | Formulation | Formulation |
| Parameters | Pilocarpine | 3A | 3B | 3C Fasted | 3D | 3E | 3C Fed |
| AUC0-t | 8.190 (50.1) | 12.06 (54.8) | 10.23 (56.5) | 10.11 (63.4) | 9.762 (48.2) | 8.814 (50.9) | 17.17 (30.3) |
| (ng*hr/mL) | [n = 16] | [n = 15] | [n = 16] | [n = 16] | [n = 16] | [n = 16] | [n = 15] |
| AUC0-inf | 8.670 (50.4) | 12.80 (56.5) | 11.01 (58.1) | 10.79 (64.4) | 10.38 (49.5) | 9.437 (52.4) | 18.70 (31.4) |
| (ng*hr/mL) | [n = 16] | [n = 15] | [n = 16] | [n = 16] | [n = 16] | [n = 16] | [n = 14] |
| Cₘₐₓ (ng/mL) | 4.239 (64.1) | 5.349 (56.2) | 4.471 (52.0) | 4.611 (68.0) | 4.220 (51.8) | 3.858 (55.1) | 3.891 (39.1) |
| | [n = 16] | [n = 15] | [n = 16] | [n = 16] | [n = 16] | [n = 16] | [n = 15] |
| Tₘₐₓ (hr) | 0.753 | 1.016 | 1.048 | 0.762 | 1.007 | 1.000 | 3.000 |
| | (0.47, 1.00) | (0.75, 2.52) | (0.75, 2.00) | (0.50, 1.50) | (0.75, 2.50) | (0.75, 2.51) | (1.00, 4.01) |
| | [n = 16] | [n = 15] | [n = 16] | [n = 16] | [n = 16] | [n = 16] | [n = 15] |
| t1/2 (hr) | 2.116 (46.8) | 2.825 (48.1) | 2.558 (49.9) | 2.693 (45.5) | 2.781 (51.3) | 2.590 (54.3) | 2.671 (25.6) |
| | [n = 16] | [n = 15] | [n = 16] | [n = 16] | [n = 16] | [n = 16] | [n = 14] |
| CL/F (L/hr) | 392.1 (50.4) | 265.6 (56.5) | 308.9 (58.1) | 315.2 (64.4) | 327.5 (49.5) | 360.3 (52.4) | 181.8 (31.4) |
| | [n = 16] | [n = 15] | [n = 16] | [n = 16] | [n = 16] | [n = 16] | [n = 14] |
| Vz/F (L) | 1197 (32.9) | 1082 (40.3) | 1140 (37.6) | 1225 (32.6) | 1314 (39.6) | 1346 (42.4) | 700.5 (35.1) |
| | [n = 16] | [n = 15] | [n = 16] | [n = 16] | [n = 16] | [n = 16] | [n = 14] |

Oxybutynin + Pilocarpine: 7.5 mg Oxybutynin Chloride and 7.5 mg Pilocarpine Hydrochloride Tablets Under Fasted Conditions (Treatment A)
DMVT-504 Formulation 3A: 7.5 mg Oxybutynin Chloride and 7.5 mg Pilocarpine Hydrochloride FDC Formulation 3A Under Fasted Conditions (Treatment B)
DMVT-504 Formulation 3B: 7.5 mg Oxybutynin Chloride and 7.5 mg Pilocarpine Hydrochloride FDC Formulation 3B Under Fasted Conditions (Treatment C)
DMVT-504 Formulation 3C Fasted: 7.5 mg Oxybutynin Chloride and 7.5 mg Pilocarpine Hydrochloride FDC Formulation 3C Under Fasted Conditions (Treatment D)
DMVT-504 Formulation 3D: 7.5 mg Oxybutynin Chloride and 7.5 mg Pilocarpine Hydrochloride FDC Formulation 3D Under Fasted Conditions (Treatment E)
DMVT-504 Formulation 3E: 7.5 mg Oxybutynin Chloride and 7.5 mg Pilocarpine Hydrochloride FDC Formulation 3E Under Fasted Conditions (Treatment F)
DMVT-504 Formulation 3C Fed: 7.5 mg Oxybutynin Chloride and 7.5 mg Pilocarpine Hydrochloride FDC Formulation 3C Under Fed Conditions (Treatment G)
Tₘₐₓ are presented as Median (Minimum, Maximum).
Other parameters are presented as Geom Mean (Geom CV %).

Table 16 presents the S-oxybutynin enantiomer data: The Cₘₐₓ was 7.413 ng/mL when the oxybutynin plus pilocarpine tablets were administered and 9.014 ng/mL when the DMVT-504 Formulation 3A capsule was administered.

TABLE 16
Summary of Plasma S-oxybutynin Following Administration of 7.5 mg Oxybutynin Chloride and
7.5 mg Pilocarpine Hydrochloride Tablets and Various FDCs Under Fed and Fasted Conditions

| | | DMVT-504 | DMVT-504 | DMVT-504 | DMVT-504 | DMVT-504 | DMVT-504 |
|---|---|---|---|---|---|---|---|
| Pharmacokinetic | Oxybutynin + | Formulation | Formulation | Formulation | Formulation | Formulation | Formulation |
| Parameters | Pilocarpine | 3A | 3B | 3C Fasted | 3D | 3E | 3C Fed |
| AUC0-t | 10.15 (56.1) | 16.39 (56.5) | 14.38 (55.6) | 13.48 (66.3) | 13.23 (50.7) | 12.04 (57.2) | 22.52 (27.8) |
| (ng*hr/mL) | [n = 16] | [n = 15] | [n = 16] | [n = 16] | [n = 16] | [n = 16] | [n = 15] |
| AUC0-inf | 10.96 (54.7) | 17.69 (60.5) | 15.47 (58.9) | 14.62 (69.3) | 14.37 (53.8) | 13.00 (61.1) | 24.65 (31.2) |
| (ng*hr/mL) | [n = 16] | [n = 15] | [n = 16] | [n = 16] | [n = 16] | [n = 16] | [n = 14] |
| Cₘₐₓ (ng/mL) | 7.413 (62.7) | 9.014 (60.9) | 7.711 (46.5) | 8.039 (60.8) | 7.137 (54.1) | 6.786 (47.7) | 5.588 (35.9) |
| | [n = 16] | [n = 15] | [n = 16] | [n = 16] | [n = 16] | [n = 16] | [n = 15] |
| Tₘₐₓ (hr) | 0.471 | 0.764 | 0.877 | 0.748 | 0.752 | 0.750 | 2.499 |
| | (0.25, 0.76) | (0.50, 2.01) | (0.50, 2.00) | (0.50, 1.00) | (0.50, 2.50) | (0.50, 1.75) | (0.76, 4.00) |
| | [n = 16] | [n = 15] | [n = 16] | [n = 16] | [n = 16] | [n = 16] | [n = 15] |
| t1/2 (hr) | 2.845 (57.4) | 4.000 (53.2) | 3.447 (51.4) | 3.870 (59.6) | 3.549 (72.9) | 3.155 (61.9) | 3.095 (33.8) |
| | [n = 16] | [n = 15] | [n = 16] | [n = 16] | [n = 16] | [n = 16] | [n = 14] |
| CL/F (L/hr) | 310.3 (54.7) | 192.1 (60.5) | 219.8 (58.9) | 232.5 (69.3) | 236.5 (53.8) | 261.6 (61.1) | 137.9 (31.2) |
| | [n = 16] | [n = 15] | [n = 16] | [n = 16] | [n = 16] | [n = 16] | [n = 14] |
| Vz/F (L) | 1274 (36.8) | 1109 (36.7) | 1093 (46.3) | 1298 (51.2) | 1211 (54.5) | 1191 (38.0) | 615.8 (31.5) |
| | [n = 16] | [n = 15] | [n = 16] | [n = 16] | [n = 16] | [n = 16] | [n = 14] |

Oxybutynin + Pilocarpine: 7.5 mg Oxybutynin Chloride and 7.5 mg Pilocarpine Hydrochloride Tablets Under Fasted Conditions (Treatment A)
DMVT-504 Formulation 3A: 7.5 mg Oxybutynin Chloride and 7.5 mg Pilocarpine Hydrochloride FDC Formulation 3A Under Fasted Conditions (Treatment B)
DMVT-504 Formulation 3B: 7.5 mg Oxybutynin Chloride and 7.5 mg Pilocarpine Hydrochloride FDC Formulation 3B Under Fasted Conditions (Treatment C)
DMVT-504 Formulation 3C Fasted: 7.5 mg Oxybutynin Chloride and 7.5 mg Pilocarpine Hydrochloride FDC Formulation 3C Under Fasted Conditions (Treatment D)
DMVT-504 Formulation 3D: 7.5 mg Oxybutynin Chloride and 7.5 mg Pilocarpine Hydrochloride FDC Formulation 3D Under Fasted Conditions (Treatment E)
DMVT-504 Formulation 3E: 7.5 mg Oxybutynin Chloride and 7.5 mg Pilocarpine Hydrochloride FDC Formulation 3E Under Fasted Conditions (Treatment F)
DMVT-504 Formulation 3C Fed: 7.5 mg Oxybutynin Chloride and 7.5 mg Pilocarpine Hydrochloride FDC Formulation 3C Under Fed Conditions (Treatment G)
Tₘₐₓ are presented as Median (Minimum, Maximum).
Other parameters are presented as Geom Mean (Geom CV %).

Table 17 presents the pilocarpine data: The Cₘₐₓ was 30.12 ng/mL when the oxybutynin plus pilocarpine tablets were administered compared to 30.88 ng/mL when the DMVT-504 Formulation 3A capsule was administered.

TABLE 17
Summary of Plasma Pilocarpine Following Administration of 7.5 mg Oxybutynin Chloride and
7.5 mg Pilocarpine Hydrochloride Tablets and Various FDCs Under Fed and Fasted Conditions

| | | DMVT-504 | DMVT-504 | DMVT-504 | DMVT-504 | DMVT-504 | DMVT-504 |
|---|---|---|---|---|---|---|---|
| Pharmacokinetic | Oxybutynin + | Formulation | Formulation | Formulation | Formulation | Formulation | Formulation |
| Parameters | Pilocarpine | 3A | 3B | 3C Fasted | 3D | 3E | 3C Fed |
| AUC0-t | 80.42 (33.8) | 84.38 (32.4) | 78.09 (38.8) | 72.79 (41.3) | 73.16 (40.9) | 67.01 (39.5) | 72.83 (35.0) |
| (ng*hr/mL) | [n = 16] | [n = 15] | [n = 16] | [n = 16] | [n = 16] | [n = 16] | [n = 15] |
| AUC0-inf | 81.15 (34.0) | 85.21 (32.4) | 78.81 (38.8) | 73.59 (41.5) | 73.84 (41.1) | 68.12 (39.7) | 71.10 (33.1) |
| (ng*hr/mL) | [n = 16] | [n = 15] | [n = 16] | [n = 16] | [n = 16] | [n = 16] | [n = 14] |
| Cₘₐₓ (ng/ml) | 30.12 (32.0) | 30.88 (27.2) | 28.80 (27.6) | 24.96 (43.6) | 24.85 (43.0) | 23.97 (30.7) | 19.96 (35.8) |
| | [n = 16] | [n = 15] | [n = 16] | [n = 16] | [n = 16] | [n = 16] | [n = 15] |
| Tₘₐₓ (hr) | 1.257 | 1.751 | 1.751 | 2.019 | 2.500 | 2.001 | 3.000 |
| | (1.25, 3.00) | (0.75, 3.01) | (1.25, 3.00) | (1.50, 3.03) | (1.25, 3.02) | (1.75, 4.00) | (2.00, 6.02) |
| | [n = 16] | [n = 15] | [n = 16] | [n = 16] | [n = 16] | [n = 16] | [n = 15] |
| t1/2 (hr) | 1.532 (14.2) | 1.500 (15.6) | 1.462 (16.1) | 1.513 (14.5) | 1.528 (9.4) | 1.477 (21.0) | 1.399 (13.5) |
| | [n = 16] | [n = 15] | [n = 16] | [n = 16] | [n = 16] | [n = 16] | [n = 14] |
| CL/F (L/hr) | 78.86 (34.0) | 75.11 (32.4) | 81.21 (38.8) | 86.96 (41.5) | 86.67 (41.1) | 93.96 (39.7) | 90.02 (33.1) |
| | [n = 16] | [n = 15] | [n = 16] | [n = 16] | [n = 16] | [n = 16] | [n = 14] |
| Vz/F (L) | 174.3 (29.1) | 162.6 (23.6) | 171.3 (31.7) | 189.8 (32.8) | 191.1 (38.9) | 200.2 (28.8) | 181.7 (28.2) |
| | [n = 16] | [n = 15] | [n = 16] | [n = 16] | [n = 16] | [n = 16] | [n = 14] |

Oxybutynin + Pilocarpine: 7.5 mg Oxybutynin Chloride and 7.5 mg Pilocarpine Hydrochloride Tablets Under Fasted Conditions (Treatment A)
DMVT-504 Formulation 3A: 7.5 mg Oxybutynin Chloride and 7.5 mg Pilocarpine Hydrochloride FDC Formulation 3A Under Fasted Conditions (Treatment B)
DMVT-504 Formulation 3B: 7.5 mg Oxybutynin Chloride and 7.5 mg Pilocarpine Hydrochloride FDC Formulation 3B Under Fasted Conditions (Treatment C)
DMVT-504 Formulation 3C Fasted: 7.5 mg Oxybutynin Chloride and 7.5 mg Pilocarpine Hydrochloride FDC Formulation 3C Under Fasted Conditions (Treatment D)
DMVT-504 Formulation 3D: 7.5 mg Oxybutynin Chloride and 7.5 mg Pilocarpine Hydrochloride FDC Formulation 3D Under Fasted Conditions (Treatment E)
DMVT-504 Formulation 3E: 7.5 mg Oxybutynin Chloride and 7.5 mg Pilocarpine Hydrochloride FDC Formulation 3E Under Fasted Conditions (Treatment F)
DMVT-504 Formulation 3C Fed: 7.5 mg Oxybutynin Chloride and 7.5 mg Pilocarpine Hydrochloride FDC Formulation 3C Under Fed Conditions (Treatment G)
Tₘₐₓ are presented as Median (Minimum, Maximum).
Other parameters are presented as Geom Mean (Geom CV %).

1. A fixed dose pharmaceutical composition comprising an oxybutynin component and a pilocarpine component,
wherein the oxybutynin component is a plurality of immediate release beads, each immediate release bead comprises:
an inert core of the immediate release bead having a diameter of about 450 μm to about 700 μm, and
a drug layer of the immediate release bead comprising oxybutynin, or a pharmaceutically acceptable salt thereof, coated on the inert core of the immediate release bead, and
wherein the pilocarpine component is a plurality of delayed-immediate release beads, each delayed-immediate release bead comprises:
an inert core of the delayed-immediate release bead having a diameter of about 250 μm to about 700 μm,
a drug layer of the delayed-immediate release bead comprising pilocarpine, or a pharmaceutically acceptable salt thereof, coated on the inert core of the delayed-immediate release bead, and
a delayed release coating of the delayed-immediate release bead coated on the drug layer of the delayed-immediate release bead,
wherein the oxybutynin component contains about 2 mg to about 10 mg of oxybutynin, or pharmaceutically acceptable salt thereof, and
wherein the pilocarpine component contains about 2 mg to about 10 mg of pilocarpine, or pharmaceutically acceptable salt thereof.

2. The fixed dose pharmaceutical composition of claim 1, wherein the plurality of immediate release beads and the plurality of delayed-immediate release beads are encapsulated in a size 3 capsule.

3. The fixed dose pharmaceutical composition of claim 1, wherein each of the immediate release beads further comprises a seal coating coated on the drug layer of the immediate release bead.

4. The fixed dose pharmaceutical composition of claim 3, wherein the seal coating of the immediate release bead is at about 0.5% to about 5.0% weight/weight of the immediate release bead.

5. The fixed dose pharmaceutical composition of claim 1, wherein the inert core of the immediate release bead is a microcrystalline cellulose sphere at about 70% to about 95% weight/weight of the immediate release bead.

6. The fixed dose pharmaceutical composition of claim 1, wherein the drug layer of the immediate release bead further comprises a binder, and an anti-tacking agent.

7. The fixed dose pharmaceutical composition of claim 6, wherein the oxybutynin, or pharmaceutically acceptable salt thereof, is about 2% to about 15% weight/weight of the immediate release bead, the binder is about 2% to about 15% weight/weight of the immediate release bead, and the anti-tacking agent is about 1% to about 10% weight/weight of the immediate release bead.

8. A pharmaceutical composition comprising:
a plurality of immediate release beads, each bead comprises:
an inert core of the immediate release bead having a diameter of about 450 μm to about 700 μm, and
a drug layer of the immediate release bead comprising oxybutynin, or a pharmaceutically acceptable salt thereof, coated on the inert core of the immediate release bead,
wherein the plurality of immediate release beads contains a total amount of about 2 mg to about 10 mg of oxybutynin, or pharmaceutically acceptable salt thereof.

9. The pharmaceutical composition of claim 8, wherein the drug layer of the immediate release bead is further coated with a seal coat.

10. The pharmaceutical composition of claim 9, wherein:
the seal coat of the immediate release bead is at about 0.5% to about 5.0% weight/weight of the immediate release bead; and
the inert core of the immediate release bead comprises a microcrystalline cellulose sphere at 70% to about 95% weight/weight of the immediate release bead.

11. The pharmaceutical composition of claim 8, wherein the drug layer of the immediate release bead further comprises a binder and an anti-tacking agent.

12. A method of treating hyperhidrosis comprising administering to a subject in need thereof a fixed dose pharmaceutical composition of claim 1.

13. The method of claim 12, wherein the hyperhidrosis is selected from the group consisting of primary (focal) hyperhidrosis, secondary hyperhidrosis, axillary hyperhidrosis, palmar hyperhidrosis, plantar hyperhidrosis, craniofacial hyperhidrosis, generalized hyperhidrosis, and compensatory sweating post-surgery.

14. The method of claim 12, wherein one or more side effects caused by oxybutynin is reduced.

15. The method of claim 14, wherein the one or more side effect includes dry mouth.
